(19) United States
(12) Patent Application Publication
(10) Pub. No.: US 2020/0157638 A1
(43) Pub. Date: May 21, 2020

(54) BIOMARKERS AND PATIENT SELECTION STRATEGIES

(71) Applicant: SIERRA ONCOLOGY, INC., Plymouth, MI (US)

(72) Inventors: Christian Andrew HASSIG, San Diego, CA (US); Bryan William STROUSE, Ann Arbor, MI (US); Ryan James HANSEN, San Diego, CA (US); Angie J. YOU, Mill Valley, CA (US)

(21) Appl. No.: 16/618,028

(22) PCT Filed: Jun. 1, 2018
(86) PCT No.: PCT/US18/35566

§ 371 Date: Nov. 27, 2019

Related U.S. Application Data
(60) Provisional application No. 62/513,954, filed on Jun. 1, 2017.
(60) Provisional application No. 62/597,759, filed on Dec. 12, 2017.
(60) Provisional application No. 62/628,900, filed on Feb. 9, 2018.
(60) Provisional application No. 62/650,199, filed on Mar. 29, 2018.

(51) Int. Cl. C12Q 1/6886; C12Q 1/6872; C12Q 1/686

(52) U.S. Cl. CPC C12Q 1/6886; C12Q 1/6872; C12Q 1/686; G01N 2333/025; C12Q 2600/106; G01N 2800/52; C12Q 2545/10; C12Q 2600/158; G01N 2800/60; C12Q 2600/156

(57) ABSTRACT

Herein disclosed are methods for treatment of cancer and methods for patient selection for administration of cancer treatment regimens comprising inhibitors of checkpoint kinase 1 (Chk1). In particular, disclosed herein are methods for identification of patients with tumors harboring genetic alterations that confer sensitivity to Chk1 inhibition.

### CROSS REFERENCE TO RELATED APPLICATIONS

This application claims the benefit of U.S. Provisional Application Nos. 62/513,954 filed Jun. 1, 2017; 62/597,759 filed Dec. 12, 2017; 62/628,900 filed Feb. 9, 2018; and 62/650,199 filed Mar. 29, 2018, all of which are hereby incorporated by reference in their entirety.

### BACKGROUND OF THE INVENTION

### Field of the Invention

This invention relates to methods for treatment of cancer and methods for patient selection for administration of cancer treatment regimens comprising administering inhibitors of checkpoint kinase 1 (Chk1).

### Description of the Related Art

Replication Stress (RS) occurs during the process of cellular DNA replication and likely contributes to genomic instability, oncogenesis and tumor progression. RS is caused by a range of factors such as complex DNA secondary structure, damaged DNA, and a limiting dNTP pool. RS can be induced by external sources, such as genotoxic agents, or internal sources, such as genetic alterations. In cancer cells, RS induced by oncogenes (e.g., MYC, RAS, CCNE1), genetic mutations in DNA repair machinery (e.g., BRCA1 or FANCA), and/or loss of function in tumor suppressors (e.g., TP53 or ATM) results in persistent DNA damage and genomic instability leading to an increased dependency on cell cycle checkpoints for survival.

The DNA Damage Response (DDR) network is a system of cellular pathways that detect DNA damage, pause the cell cycle, and repair damaged DNA to restore genomic integrity. Checkpoint kinase 1 (Chk1) is a key regulator of important cell cycle checkpoints and a central mediator of the DDR network. Chk1 plays a critical role in the response to RS and DNA damage by mediating S and G2/M cell cycle arrest and homologous recombination repair, as well as by stabilizing replication forks and regulating origin firing in response to stalled replication.

SRA737 (formerly CCT245737), a Chk1 inhibitor, is a potent, highly selective, orally bioavailable inhibitor of with excellent pharmaceutical properties (IC₅₀: Chk1=1.4 nM). SRA737 is disclosed and claimed in U.S. Pat. No. 9,663,503, the contents of which are incorporated herein in its entirety. SRA737 demonstrates robust efficacy in various preclinical or murine models as a single agent and in combination with selected cytotoxics and other anticancer agents; however, improved methods for the use of Chk1 inhibitors for the treatment of cancer and improved methods for the identification of patients with cancer that are responsive to treatment regimens comprising Chk1 inhibitors are needed.

### SUMMARY OF THE INVENTION

In certain aspects, described herein are methods of treating a tumor in an individual having a cancer, the method comprising: administering a Chk1 inhibitor to the individual, wherein the tumor is identified as having genetic alterations that confer high levels of replication stress and thereby sensitivity to the Chk1 inhibitor by synthetic lethality; and wherein the genetic alterations are at least two of property a, property b, property c, or property d wherein; property a is a gain of function mutation or amplification or overexpression of at least one oncogenic driver gene or other gene implicated in Chk1 pathway sensitivity; property b is a loss of function or deleterious mutation in at least one DNA damage repair (DDR) pathway gene implicated in Chk1 pathway sensitivity; property c is a gain of function mutation or amplification of at least one replication stress gene implicated in Chk1 pathway sensitivity; and property d is a deleterious mutation in a tumor suppressor (TS) gene implicated in Chk1 pathway sensitivity. In certain embodiments, the genetic alterations are property d and at least one of property a, property b, or property c. In certain embodiments, the methods, further comprise determining whether or not the tumor comprises the property a, property b, property c or property d. In certain embodiments, the property a, property b, property c, or property d are determined by using Next-Generation Sequencing (NGS), by immunohistochemistry, by mass spectrometry (MS), by liquid chromatograph mass spectrometry (LC-MS), by quantitative PCR, by RNA sequencing (RNAseq) or by fluorescence activated cell sorting (FACS) analysis. In certain embodiments, the property a, property b, property c or property d are determined using NGS. In certain embodiments, the tumor suppressor gene is RB1, TP53 or ATM. In certain embodiments, the tumor suppressor gene is RAD50, FBXW7, PARK2, CDKN2A or CDKN2B. In certain embodiments, property d is established by establishing positivity for human papillomavirus (HPV). In certain embodiments, the cancer is a squamous cell carcinoma. In certain embodiments, the squamous cell carcinoma is head and neck squamous cell carcinoma, cervical cancer or anogenital squamous cell carcinoma. In certain embodiments, the oncogenic driver gene is MYC, MYCN or CCNE1. In certain embodiments, the DDR pathway gene is ATM, BRCA1, BRCA2 or an FA pathway gene. In certain embodiments, the DDR pathway gene is MRE11A or ATR. In certain embodiments, property b is established by establishing a microsatellite instability or deficiency in mismatch repair (MMR). In certain embodiments, the cancer is colorectal cancer (CRC) or endometrial cancer. In certain embodiments, the replication stress gene is ATR or CHEK1. In certain embodiments, the methods further comprise chemotherapy, a treatment comprising administering an antibody, antibody fragment, antibody drug conjugate or radiation treatment. In certain embodiments, the methods further comprising administering an external inducer of replication stress. In certain embodiments, the methods further comprise administering gemcitabine, hydroxyurea, a ribonucleotide reductase inhibitor, cisplatin, etoposide, SN-38/CPT-11, mitomycin C, an inhibitor of ATR, an inhibitor of PARP or combinations thereof. In certain embodiments, the method further comprises administering gemcitabine. In certain embodiments, the individual has a cancer selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer. In certain embodiments, the Chk1 inhibitor is SRA737.

In certain aspects described here are methods of treating a tumor in an individual having a cancer, the method comprising: administering a Chk1 inhibitor and a genotoxic agent that confers increased levels of replication stress and thereby to the individual, wherein the tumor is identified as having at least one genetic alteration that confers high levels of replication stress and thereby sensitivity to the Chk1 inhibitor by synthetic lethality; and wherein the genetic alteration is at least one of property a, property b, property c, or property d wherein: property a is a gain of function mutation or amplification or overexpression of at least one oncogenic driver gene or other gene implicated in Chk1 pathway sensitivity; property b is a loss of function or deleterious mutation in at least one DNA damage repair (DDR) pathway gene implicated in Chk1 pathway sensitivity; property c is a gain of function mutation or amplification of at least one replication stress gene implicated in Chk1 pathway sensitivity; and property d is a deleterious mutation in a tumor suppressor (TS) gene implicated in Chk1 pathway sensitivity. In certain embodiments, the method, further comprises determining whether or not the tumor comprises the at least one of the property a, property b, property c, or property d. In certain embodiments, the property a, property b, property c, or property d are determined by using Next-Generation Sequencing (NGS), by immunohistochemistry, by mass spectrometry (MS), by liquid chromatograph mass spectrometry (LC-MS), by quantitative PCR, by RNA sequencing (RNAseq) or by fluorescence activated cell sorting (FACS) analysis. In certain embodiments, at least one of the property a, property b, property c, or property d is determined using Next-Generation Sequencing (NGS). In certain embodiments, the tumor suppressor gene is RB1, TP53 or ATM. In certain embodiments, the tumor suppressor gene is RAD50, FBXW7 or PARK2. In certain embodiments, property d is established by establishing positivity for HPV. In certain embodiments, the cancer is a squamous cell carcinoma. In certain embodiments, the squamous cell carcinoma is head and neck squamous cell carcinoma, cervical cancer or anogenital squamous cell carcinoma. In certain embodiments, the oncogenic driver gene is MYC, MYCL, MYCN or CCNE1. In certain embodiments, the DDR pathway gene is ATM, BRCA1, BRCA2 or an FA pathway gene. In certain embodiments, the DDR pathway gene is MRE11A or ATR. In certain embodiments, property b is established by establishing microsatellite instability or deficiency in mismatch repair (MMR). In certain embodiments, the cancer is colorectal cancer (CRC). In certain embodiments, the replication stress gene is ATR or CHEK1. In certain embodiments, the method further comprises chemotherapy, a treatment comprising administering an antibody, antibody fragment, antibody drug conjugate or radiation treatment. In certain embodiments, the method further comprises administering an external inducer of replication stress. In certain embodiments, the genotoxic agent is gemcitabine, hydroxyurea, cisplatin, a ribonucleotide reductase inhibitor, etoposide, SN-38/CPT-11, mitomycin C, an inhibitor of ATR, an inhibitor of PARP or combinations thereof. In certain embodiments, the genotoxic agent is gemcitabine. In certain embodiments, the individual has a cancer selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer. In certain embodiments, the Chk1 inhibitor is SRA737.

In certain aspects, described herein are methods of treating a tumor in an individual having cancer, the method comprising administering a Chk1 inhibitor to the individual, wherein the tumor, the germline or combinations thereof is characterized by having wild type BRCA1 or BRCA2 and is resistant or refractory to platinum based chemotherapy. In certain embodiments, described herein is a method of treating a tumor in an individual having cancer, the method comprising administering a Chk1 inhibitor to the individual, wherein the individual has been previously treated with a PARP inhibitor on the basis of a mutation in at least one homologous recombination repair gene. In certain embodiments, the cancer is ovarian cancer. In certain embodiments, the tumor is further characterized by a mutation, optionally a deleterious mutation, in a tumor suppressor gene. In certain embodiments, the tumor suppressor gene is RB1, TP53, ATM, RAD50, FBXW7 or PARK2. In certain embodiments, the method further comprises determining whether or not the tumor comprises the tumor suppressor gene mutation. In certain embodiments, the tumor is further characterized by CCNE1 gene overexpression; and wherein the CCNE1 gene overexpression is at least one of overexpression of Cyclin E1 (CCNE1), CCNE1 gene amplification, CCNE1 gene copy number gain, CCNE1 mRNA overexpression and Cyclin E protein overexpression. In certain embodiments, the CCNE1 gene overexpression is increased mRNA levels, Cyclin E protein levels or combinations thereof compared to an at least one reference sample. In certain embodiments, the CCNE1 gene overexpression is detected by immunohistochemistry (IHC) by mass spectrometry (MS) or by liquid chromatography mass spectrometry (LC-MS). In certain embodiments, the CCNE1 gene overexpression is caused by CCNE1 gene amplification or alternative genetic alteration with similar functional effect. In certain embodiments, the CCNE1 gene amplification or alternative genetic alteration is detected by NGS. In certain embodiments, the method further comprises characterizing the CCNE1 gene overexpression, optionally by using NGS, by immunohistochemistry (IHC), by mass spectrometry (MS), by liquid chromatograph mass spectrometry (LC-MS), by quantitative PCR, by RNAseq or by FACS analysis or by determination of CyclinE-CDK2 activity. In certain embodiments, the characterization of the CCNE1 gene overexpression is performed by detecting circulating RNA or circulating DNA. In certain embodiments, the determination of CyclinE-CDK2 activity is detecting phosphorylation of CyclinE-CDK2 substrates. In certain embodiments, the CyclinE-CDK2 substrate is MCM2, retinoblastoma protein (Rb), p27, p21, Smad3, CBP/p300, E2F-5, p220(NPAT) or FOXO1. In certain embodiments, the method further comprises chemotherapy, a treatment comprising administering an antibody, antibody fragment, antibody drug conjugate or radiation treatment. In certain embodiments, the method further comprises administration of an external inducer of replication stress. In certain embodiments, the method further comprises administration of gemcitabine, hydroxyurea, cisplatin, etoposide, SN-38/CPT-11, mitomycin C, an inhibitor of ATR, an inhibitor of PARP or combinations thereof. In certain embodiments, the method further comprises administration of gemcitabine. In certain embodiments, the individual has a cancer selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer. In certain embodiments, the cancer is ovarian cancer. In certain embodiments, the ovarian cancer is high grade serous ovarian cancer (HGSOC). In certain embodiments, the HGSOC is resistant or refractory to platinum-based chemotherapy. In certain embodiments, the HGSOC is deficient in homologous recombination or proficient in homologous recombination. In certain embodiments, the Chk1 inhibitor is SRA737.

In certain aspects, disclosed herein are methods of treating a tumor in an individual having colorectal cancer, wherein the tumor is characterized by microsatellite instability or a deficiency in mismatch repair, the method comprising administering a Chk1 inhibitor to the individual.

In certain aspects, described herein is a method of treating a tumor in an individual having endometrial cancer, wherein the tumor is characterized by microsatellite instability or having a mismatch repair deficiency, the method comprising administering a Chk1 inhibitor to the individual

In certain aspects, disclosed herein are methods of treating a tumor in an individual having squamous cell carcinoma, wherein the individual is HPV positive, the method comprising administering a Chk1 inhibitor to the individual. In certain embodiments, the squamous cell carcinoma is head and neck squamous cell carcinoma, cervical cancer, or anogenital squamous cell carcinoma. In certain embodiments, the tumor is further characterized by at least one of property a, property b, or property c, wherein: property a is a gain of function mutation or amplification of at least one oncogenic driver gene or other gene implicated in Chk1 pathway sensitivity; property b is a loss of function or deleterious mutation in at least one DNA damage repair (DDR) pathway gene implicated in Chk1 pathway sensitivity; and property c is a gain of function mutation or amplification of at least one replication stress gene implicated in Chk1 pathway sensitivity.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIG. 1 is a diagram that illustrates genomic alterations of cancer cells conferring synthetic lethality by inhibition of Chk1.

FIG. 2 is a diagram of combinations of genetic alterations of different categories of genes, wherein one of the categories is a mutation in a tumor suppressor gene, according ton an embodiment of the invention.

FIG. 3 is a diagram illustrating cancer cell sensitivity to Chk1 inhibition wherein cancer cells with genetic alterations exhibit replication stress induced by oncogene activation or a genotoxic agent, according to an embodiment of the invention.

FIG. 4 are graphs depicting growth inhibition of cancer cell lines to Chk1 inhibition by SRA737.

FIG. 5 are graphs depicting sensitivity of ovarian cancer cell lines, OVCAR-3 and OVCAR-5, harboring gene amplification or gene overexpression, respectively, to Chk1 inhibition by SRA737.

FIG. 6A is a graph showing reduced tumor volume in a mouse OVCAR-3 xenograft model treated with SRA737.

FIG. 6B is a graph showing change in body weight in the OVCAR-3 xenograft model treated with SRA737.

FIG. 7A is a graph showing reduced tumor volume in a mouse OVCAR-3 xenograft model treated with SRA737, the PARP inhibitor, Olaparib, or SRA737 and Olaparib.

FIG. 7B is a graph showing change in body weight in the OVCAR-3 xenograft model treated with SRA737 the PARP inhibitor, Olaparib, or SRA737 and Olaparib.

FIG. 8 is a diagram of a clinical trial design for administration of SRA737 in patient cohorts with tumors expected to have sensitivity to Chk1 inhibition, according to an embodiment of the invention.

### DETAILED DESCRIPTION OF THE PREFERRED EMBODIMENTS

### Methods of the Invention

Disclosed herein are methods for inhibiting tumor growth in a subject, e.g., a human, by administration of a Chk1 inhibitor, wherein the Chk1 inhibitor interferes with the biological role of Chk1 and induces synthetic lethality in selected genetically-mutated cancer cells. In certain embodiments, disclosed herein are methods for the treatment of cancer by administration of the Chk1 inhibitor, SRA737. A detailed description of the SRA737 compound and methods of use thereof are found below.

Methods of Treating a Tumor with Genetic Alterations

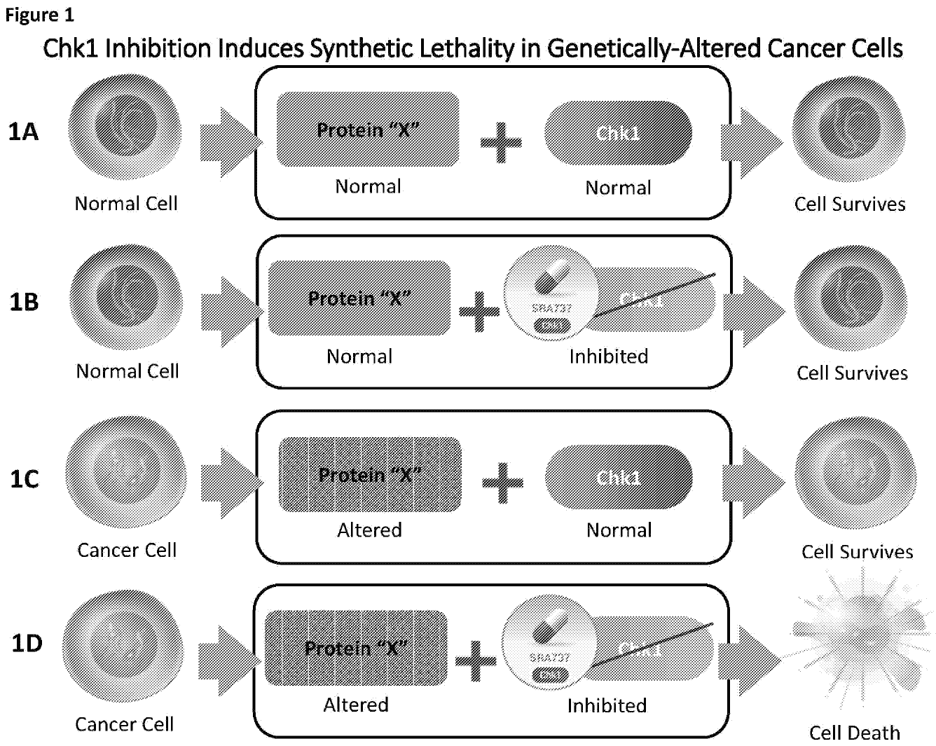

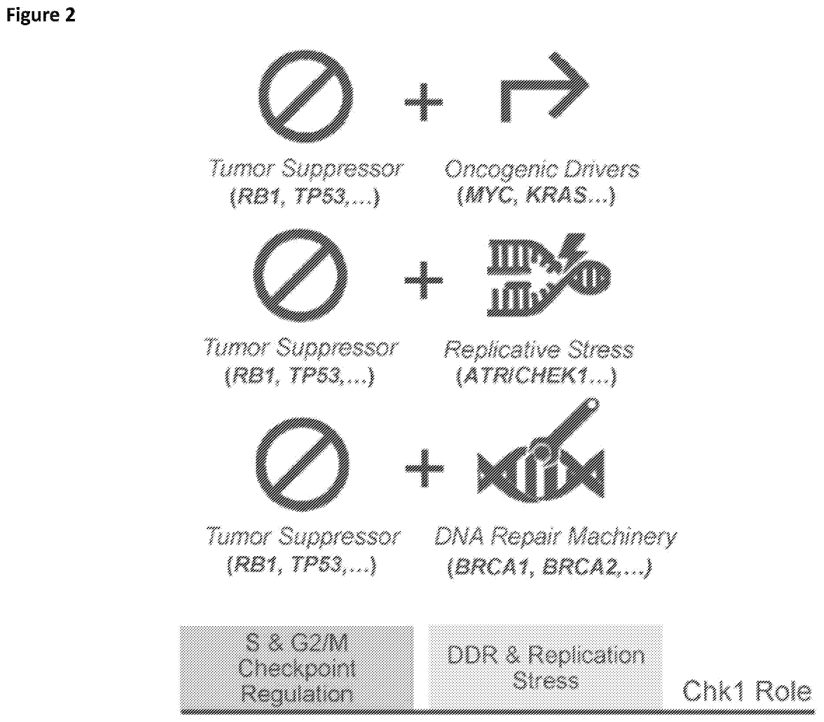

In certain aspects, disclosed herein are methods of treating a tumor in an individual having a cancer, the method comprising administering a Chk1 inhibitor to the individual wherein the tumor is identified as has genetic alterations that confer sensitivity to the Chk1 inhibitor by synthetic lethality (FIG. 1). In certain aspects, the genetic alterations comprise at least two of a gain of function mutation or amplification or overexpression of an oncogenic driver gene, a loss of function or deleterious mutation in a DDR pathway gene, a gain of function mutation or amplification of a replication stress gene, a loss of function or deleterious mutation of a replication stress gene, and a deleterious mutation in tumor suppressor gene (Table 1). In certain embodiments, the genetic alterations comprise a deleterious mutation in tumor suppressor gene and at least one of a gain of function mutation or amplification or overexpression of an oncogenic driver gene, a loss of function or deleterious mutation in a DDR pathway gene, and a gain of function mutation or amplification of a replication stress gene (FIG. 2). In certain embodiments, the genetic alterations comprise at least two of: a gain of function mutation or amplification or overexpression of an oncogenic driver gene, a loss of function or deleterious mutation in a DDR pathway gene, a gain of function mutation or amplification of a replication stress gene, and positivity for human papillomavirus (HPV). In certain embodiments, the genetic alterations comprise: positivity for human papillomavirus (HPV), and at least one of: a gain of function mutation or amplification or overexpression of an oncogenic driver gene, a loss of function or deleterious mutation in a DDR pathway gene, and a gain of function mutation or amplification of a replication stress gene. In certain embodiments, the genetic alterations comprise microsatellite instability and/or deficiency in mismatch repair (MMR) and at least one of: a gain of function mutation, amplification or overexpression of an oncogenic driver gene, a gain of function mutation or amplification of a replication stress gene, and a deleterious mutation in tumor suppressor gene.

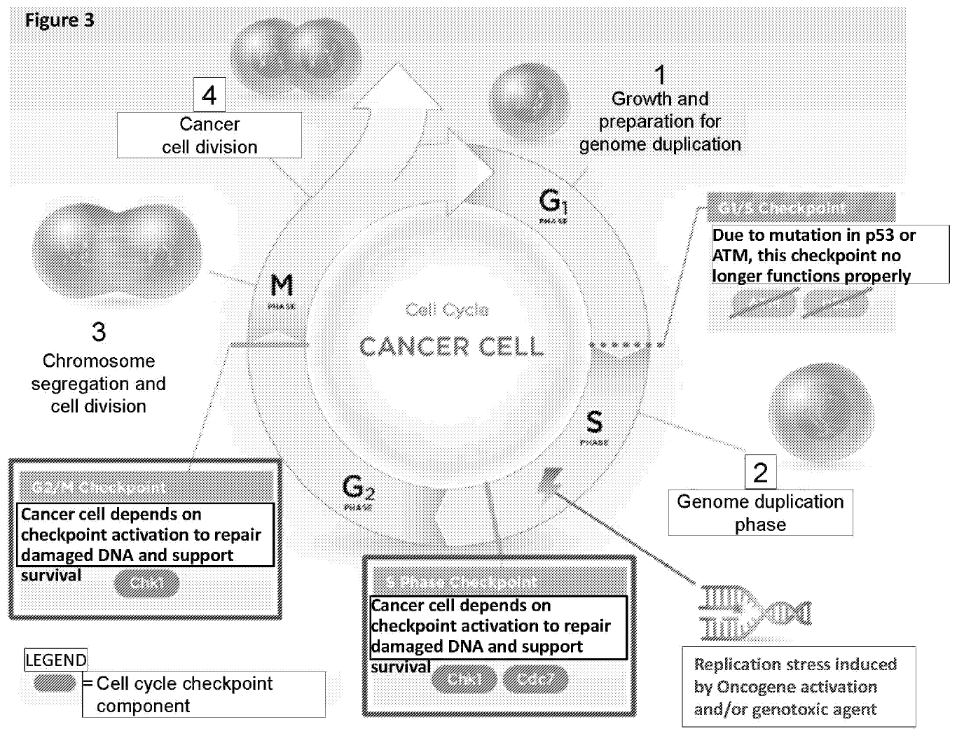

In certain aspects, provided herein are methods of treating a tumor in an individual comprising administering a Chk1 inhibitor and a genotoxic chemotherapeutic agent that confers increased levels of replication stress to the individual, wherein the tumor is identified as having genetic alterations that confer high levels of replication stress and thereby sensitivity to the Chk1 inhibitor by synthetic lethality (FIG. 3). In certain aspects, the genetic alterations comprise at least one of a gain of function mutation or amplification or overexpression of an oncogenic driver gene, a loss of function or deleterious mutation in a DDR pathway gene, a gain of function mutation or amplification of a replication stress gene, and a deleterious mutation in tumor suppressor gene.

In certain aspects, described herein are methods of treating a tumor in an individual having cancer, the method comprising administering a Chk1 inhibitor to the individual, wherein the tumor is characterized by having wild type BRCA1 and is resistant or refractory to platinum based chemotherapy. In certain aspects, described herein is a method of treating a tumor in an individual having cancer, the method comprising administering a Chk1 inhibitor to the individual, wherein the tumor is characterized by having a mutation in BRCA1 and the individual has been previously treated with a PARP inhibitor.

In certain aspects, provided herein are methods of treating a tumor in an individual, comprising administering a Chk1 inhibitor to the individual, wherein the tumor is characterized by overexpression of Cyclin E1 (CCNE1). In certain embodiments, the tumor is further characterized by a deleterious mutation in a tumor suppressor gene.

TABLE 1

| Gene Class | Biological Rationale |
|---|---|
| Tumor Suppressors | Defective G1/2 checkpoint should increase |
| | reliance on remaining Chk1-regulated DNA |
| | damage checkpoints. |
| Oncogenic Drivers | Oncogene-induced hyperproliferation and |
| | cell cycle dysregulation contribute to |
| | replication stress and could increase reliance |
| | on Chk1. |
| Replicative Stress | Amplification of genes encoding ATR or |
| | Chk1 suggests greater reliance on Chk1 |
| | pathway to accommodate replication stress. |
| DNA Repair Machinery | Mutated DNA repair genes result in |
| | excessive DNA damage, and may increase |
| | reliance on Chk1-mediated DNA repair |
| | and/or cell cycle arrest functions. |

### Identification of Tumor Genetic Alterations

In certain embodiments, provided herein are methods of treating a tumor with genetic alterations (e.g., a gain of function mutation or amplification or overexpression of a oncogenic driver gene, a loss of function or deleterious mutation in a DDR pathway gene, a gain of function mutation or amplification of a replication stress gene, and/or a deleterious mutation in tumor suppressor gene), the methods comprising determining whether or not the tumor has the genetic alterations. The genetic alterations can be identified using any method known in the art for determining gene alterations, mRNA alterations, mRNA expression changes, protein alterations, and/or protein expression changes. Examples of methods that can be used to identify the genetic alterations include, but are not limited to, Next-Generation Sequencing (NGS), quantitative PCR, mass spectrometry (MS), liquid chromatography-mass spectrometry (LC-MS), RNA sequencing (RNAseq), fluorescence activated cell sorting (FACS) analysis or immunohistochemistry. In certain embodiments, characterization of CCNE1 gene overexpression may be performed by, but not limited to, NGS, IHC, quantitative PCR, RNAseq, FACS analysis or by determination of CyclinE-CDK2 activity. In certain aspects, determination of CyclinE-CDK2 activity may be performed by detecting phosphorylation of CyclinE-CDK2 substrates such as, but not limited to, MCM2 or retinoblastoma protein (Rb) or p27 or p21 or Smad3 or CBP/p300 or E2F-5 or p220(NPAT) or FOXO1. The genetic alterations can be identified from any tumor cell sample (e.g., circulating tumor cells from blood or plasma samples and biopsied tumor samples).

### Tumor Inhibition

The present disclosure is directed to methods using inhibitors of Chk1 to inhibit the progression of, reduce the size of, the aggregation of, reduce the volume of, and/or otherwise inhibit the growth of a tumor. Also provided herein are methods of treating the underlying disease, e.g., cancer, and extending the survival of the subject.

In an embodiment provided for is a method of inhibiting the growth of a tumor in a subject in need thereof, the method comprising administering to the subject an effective amount of a Chk1 inhibitor. In some aspects, the disclosure provides for a method of inhibiting the growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by tumor volume. In some aspects, the disclosure provides for a method of inhibiting the growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the absolute size of the tumor. In some aspects, the disclosure provides for a method of inhibiting the growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the expression levels of tumor markers for that type of tumor.

### Types of Tumors

In certain aspects, the disclosed herein are methods for inhibiting growth of tumor types that are known to have a high prevalence of genomic aberrations expected to sensitize the tumor to Chk1 inhibition. In some aspects, the present disclosure provides for methods of inhibiting the growth of a tumor wherein the tumor is from a cancer that is colorectal cancer, ovarian cancer, high grade serous ovarian cancer (HGSOC), non-small cell lung cancer, small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, myeloma, brain cancer, neuroblastoma, squamous cell carcinoma, head and neck squamous cell carcinoma, anogenital squamous cell carcinoma, anogenital cancer, pancreatic cancer, and sarcoma.

Accordingly, the present disclosure also provides for methods of treating a cancer in a subject in need thereof, the method comprising administering an effective amount of a Chk1 inhibitor to the subject. In some aspects, methods are disclosed for the treatment of cancer wherein the cancer is colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma. In certain aspects, the present disclosure provides for methods for the treatment of any cancer, including but not limited to, advanced solid tumors (e.g., metastatic colorectal cancer (CRC), platinum-resistant or intolerant HGSOC, advanced NSCLC, and metastatic castration-resistant prostate cancer (mCRPC)). In certain embodiments, the present disclosure provides for methods for the treatment of breast cancer, including but not limited to triple-negative breast cancer, invasive breast cancer, metastatic breast cancer, HER2 positive breast cancer and inflammatory breast cancer.

In certain embodiments, the cancer has an amplification or overexpression of CCNE1. Germline and/or tumor wild-type BRCA1 and/or BRCA2 (i.e., BRCA1/2) has been reported to be indicative of CCNE1 amplification or overexpression (Lee, J. et al., Lancet Oncology, Jan. 18, 2018); therefore, in certain embodiments, the cancer is from a subject with wild-type germline BRCA1/2, and/or a tumor of the subject has wild-type BRCA1/2. In the description below of cancers with particular genetic alterations, for the cancers described to have CCNE1 amplification or overexpression, the genetic alteration of CCNE1 amplification or overexpression can be substituted with germline and/or tumor wild-type BRCA1/2.

In certain aspects, the present disclosure provides for methods of treating cancer, wherein the cancer is HGSOC. In certain embodiments, the cancer is HGSOC, and the HGSOC has an amplification or overexpression of CCNE1. In certain embodiments, the cancer is HGSOC, the HGSOC has an amplification or overexpression of CCNE1, and the HGSOC has a deleterious mutation in TP53. In certain embodiments, the cancer is HGSOC, and the HGSOC has an amplification or overexpression of CCNE1 and MYC. In certain embodiments, the cancer is HGSOC, the HGSOC has an amplification or overexpression of CCNE1 and MYC, and the HGSOC has a deleterious mutation in TP53. In certain embodiments, the cancer is HGSOC, and the HGSOC has an amplification or overexpression of MYC. In certain embodiments, the cancer is HGSOC; the HGSOC has an amplification or overexpression of MYC, and the HGSOC has a deleterious mutation in TP53. In certain embodiments, the cancer is HGSOC, the HGSOC has an amplification or overexpression of MYC, and the HGSOC has a deleterious mutation in RB1. In certain embodiments, the cancer is HGSOC, the HGSOC has an amplification or overexpression of MYC, and the HGSOC has a deleterious mutation in TP53 and RB1. In certain embodiments, the cancer is HGSOC, and the HGSOC has an amplification or overexpression of MYCL. In certain embodiments, the cancer is HGSOC; the HGSOC has an amplification or overexpression of MYCL, and the HGSOC has a deleterious mutation in TP53. In certain embodiments, the cancer is HGSOC, and the HGSOC has a deleterious mutation in RB1. In certain embodiments, the cancer is HGSOC, and the HGSOC has a deleterious mutation in RB and TP53. In certain embodiments, the cancer is HGSOC; and the HGSOC has a deleterious mutation or loss of function mutation in BRCA1 or BRCA2. In certain embodiments, the cancer is HGSOC; the HGSOC has a deleterious mutation or loss of function mutation in BRCA1 or BRCA2, and the HGSOC has a deleterious mutation in TP53. In certain embodiments, the cancer is HGSOC; the HGSOC has a deleterious mutation or loss of function mutation in BRCA1 or BRCA2, and the HGSOC has an amplification or overexpression of MYC. In certain embodiments, the cancer is HGSOC; the HGSOC has a deleterious mutation or loss of function mutation in BRCA1 or BRCA2, and the HGSOC has a deleterious mutation in RB1. In certain embodiments, the cancer is HGSOC; the HGSOC has a deleterious mutation or loss of function mutation in BRCA1 or BRCA2, the HGSOC has an amplification or overexpression of MYC, and the HGSOC has a deleterious mutation in TP53. In certain embodiments, the cancer is HGSOC; and the HGSOC has an amplification or overexpression CDK12. In certain embodiments, the cancer is HGSOC; the HGSOC has an amplification or overexpression CDK12, and the HGSOC has a deleterious mutation in TP53. In certain embodiments, the cancer is HGSOC; and the HGSOC has an amplification or overexpression CDK12 and MYC. In certain embodiments, the cancer is HGSOC; the HGSOC has an amplification or overexpression CDK12 and MYC, and the HGSOC has a deleterious mutation in TP53. In certain embodiments, the cancer is HGSOC, and the HGSOC has a deleterious mutation in TP53.

In certain aspects, the present disclosure provides for methods of treating cancer, wherein the cancer is metastatic castration-resistant prostate cancer (mCRPC). In certain embodiments, the cancer is mCRPC, and the mCRPC has an amplification or overexpression of MYC. In certain embodiments, the cancer is mCRPC, and the mCRPC has a deleterious mutation in TP53. In certain embodiments, the cancer is mCRPC, and the mCRPC has a deleterious mutation in RB1. In certain embodiments, the cancer is mCRPC, the mCRPC has an amplification or overexpression of MYC, and the CRPC has a deleterious mutation in TP53. In certain embodiments, the cancer is mCRPC, the mCRPC has an amplification or overexpression of MYC, and the CRPC has a deleterious mutation in RB1. In certain embodiments, the cancer is mCRPC, the mCRPC has an amplification or overexpression of MYC, and the CRPC has a deleterious mutation in PTEN. In certain embodiments, the cancer is mCRPC, and the mCRPC has a deleterious mutation in TP53 and RB1. In certain embodiments, the cancer is mCRPC, and the mCRPC has a deleterious mutation in RB1 and PTEN. In certain embodiments, the cancer is mCRPC, the mCRPC has a deleterious mutation in RB1, and the mCRPC has a deleterious mutation or loss of function mutation in BRCA2. In certain embodiments, the cancer is mCRPC, the mCRPC has a deleterious mutation in TP53 and a deleterious mutation or loss of function mutation in BRCA1. In certain embodiments, the cancer is mCRPC, and the mCRPC has a deleterious mutation or loss of function mutation in BRCA2. In certain embodiments, the cancer is mCRPC, and the mCRPC has a deleterious mutation or loss of function mutation in ATM. In certain embodiments, the cancer is mCRPC, and the mCRPC has a deleterious mutation in PTEN. In certain embodiments, the cancer is mCRPC, and the mCRPC has a deleterious mutation in PTEN and TP53.

In certain aspects, the present disclosure provides for methods of treating cancer, wherein the cancer is head and neck squamous cell carcinoma (HNSCC). In certain embodiments, the cancer is HNSCC and the HNSCC is HPV positive. In certain embodiments, the cancer is HNSCC, and the HNSCC is has a deleterious mutation in CDKN2A. In certain embodiments, the cancer is HNSCC, and the HNSCC is has a deleterious mutation in CDKN2B. In certain embodiments, the cancer is HNSCC, the HNSCC is HPV positive, and the cancer has an amplification or overexpression in PI3KCA. In certain embodiments, the cancer is HNSCC, the HNSCC is HPV positive, and the HNSCC has a deleterious mutation or loss of function mutation in ATM. In certain embodiments, the cancer is HNSCC, and the HNSCC has a deleterious mutation or loss of function mutation in CDKN2A and CDKN2B. In certain embodiments, the cancer is HNSCC, the HNSCC is has a deleterious mutation or loss of function mutation in CDKN2A and/or CDKN2B, and the HNSCC has a deleterious mutation in TP53. In certain embodiments, the cancer is HNSCC, the HNSCC has a deleterious mutation or loss of function mutation in CDKN2A and/or CDKN2B, and the HNSCC has an amplification or overexpression in MYC. In certain embodiments, the cancer is HNSCC, the HNSCC has a deleterious mutation or loss of function mutation in CDKN2A and/or CDKN2B, and the HNSCC has an amplification or overexpression in PI3KCA. In certain embodiments, the cancer is HNSCC, the HNSCC has a deleterious mutation or loss of function mutation in CDKN2A and/or CDKN2B, and the HNSCC has an amplification or overexpression in MYC. In certain embodiments, the cancer is HNSCC, the HNSCC has a deleterious mutation or loss of function mutation in CDKN2A and/or CDKN2B, the HNSCC has an amplification or overexpression in MYC, and the HNSCC has a deleterious mutation in TP53. In certain embodiments, the cancer is HNSCC, the HNSCC has a deleterious mutation or loss of function mutation in CDKN2A and/or CDKN2B, the HNSCC has an amplification or overexpression in PI3KCA, and the HNSCC has a deleterious mutation in TP53. In certain embodiments, the cancer is HNSCC, and the HNSCC has an amplification or over expression in CCNE1. In certain embodiments, the cancer is HNSCC, the HNSCC has an amplification or over expression in CCNE1, and the HNSCC has a deleterious mutation in TP53. In certain embodiments, the cancer is HNSCC, and the HNSCC has deleterious mutation in FBXW7. In certain embodiments, the cancer is HNSCC, the HNSCC has deleterious mutation in FBXW7, and the HNSCC has a deleterious mutation in TP53. In certain embodiments, the cancer is HNSCC, and the HNSCC has deleterious mutation in PARK2. In certain embodiments, the cancer is HNSCC, the HNSCC has deleterious mutation in PARK2, and the HNSCC has a deleterious mutation in TP53. In certain embodiments, the cancer is HNSCC, and the HNSCC has an amplification or overexpression in MYC. In certain embodiments, the cancer is HNSCC, the HNSCC has an amplification or overexpression in MYC, and the HNSCC has deleterious mutation or loss of function mutation in ATM. In certain embodiments, the cancer is HNSCC, and the HNSCC has an amplification or overexpression in MYC and PIK3CA. In certain embodiments, the cancer is HNSCC, the HNSCC has an amplification or overexpression in MYC, and the HNSCC has a deleterious mutation in TP53. In certain embodiments, the cancer is HNSCC, the HNSCC has an amplification or overexpression in MYC and PIK3CA, and the HNSCC has a deleterious mutation in TP53. In certain embodiments, the cancer is HNSCC, and the HNSCC has a deleterious mutation or loss of function mutation in ATM. In certain embodiments, the cancer is HNSCC, and the HNSCC has a deleterious mutation in TP53. In certain embodiments, the cancer is HNSCC, and the HNSCC has an amplification or overexpression PIK3CA.

In certain aspects, the present disclosure provides for methods of treating cancer, wherein the cancer is colorectal cancer (CRC). In certain embodiments, the cancer is CRC, and the CRC has an amplification or overexpression of CCNE1. In certain embodiments, the cancer is CRC, the CRC has an amplification or overexpression of CCNE1, and the CRC has a deleterious mutation in TP53. In certain embodiments, the cancer is CRC and the CRC has a deleterious mutation in FBXW7. In certain embodiments, the cancer is CRC, and the CRC has a deleterious mutation in FBXW7 and TP53. In certain embodiments, the cancer is CRC, the CRC has a deleterious mutation in FBXW7, and the CRC has an amplification or overexpression PIK3CA. In certain embodiments, the cancer is CRC and the CRC has a deleterious mutation in PARK2. In certain embodiments, the cancer is CRC, and the CRC has a deleterious mutation in PARK2 and TP53. In certain embodiments, the cancer is CRC, and the CRC has a deleterious mutation or loss of function mutation in ATM. In certain embodiments, the cancer is CRC, the CRC has a deleterious mutation or loss of function mutation in ATM, and the CRC has a deleterious mutation in TP53. In certain embodiments, the cancer is CRC, and the CRC has an amplification or overexpression in MYC. In certain embodiments, the cancer is CRC, the CRC has an amplification or overexpression in MYC, and the CRC has a deleterious mutation in TP53. In certain embodiments, the cancer is CRC, and the CRC has a deleterious mutation in TP53. In certain embodiments, the cancer is CRC, and the CRC has an amplification or overexpression PIK3CA. In certain embodiments, the cancer is CRC, the CRC has an amplification or overexpression PIK3CA, and the CRC has a deleterious mutation in TP53.

In certain aspects, the present disclosure provides for methods of treating cancer, wherein the cancer is non-small cell lung cancer (NSCLC). In certain embodiments, the cancer is NSCLC, and the NSCLC has an amplification or overexpression of CCNE1. In certain embodiments, the cancer is NSCLC, and the NSCLC has an amplification or overexpression of CCNE1 and MYC. In certain embodiments, the cancer is NSCLC, the NSCLC has an amplification or overexpression of CCNE1, and the NSCLC has a deleterious of loss of function mutation in CDKN2A. In certain embodiments, the cancer is NSCLC, the NSCLC has an amplification or overexpression of CCNE1, and the NSCLC has a deleterious of loss of function mutation in CDKN2B. In certain embodiments, the cancer is NSCLC, the NSCLC has an amplification or overexpression of CCNE1, and the NSCLC has a deleterious mutation in TP53. In certain embodiments, the cancer is NSCLC, the NSCLC has an amplification or overexpression of CCNE1, and the NSCLC has a deleterious mutation in RB1. In certain embodiments, the cancer is NSCLC, the NSCLC has an amplification or overexpression of CCNE1 and MYC, and the NSCLC has a deleterious mutation in TP53. In certain embodiments, the cancer is NSCLC, the NSCLC has an amplification or overexpression of CCNE1, and the NSCLC has a deleterious mutation in TP53 and RB1. In certain embodiments, the cancer is NSCLC, the NSCLC has an amplification or overexpression of CCNE1, the NSCLC has a deleterious mutation in TP53, and the NSCLC has a deleterious mutation or loss of function mutation in CDKN2A and CDKN2B. In certain embodiments, the cancer is NSCLC, and the NSCLC has a deleterious mutation in PARK2. In certain embodiments, the cancer is NSCLC, the NSCLC has a deleterious mutation in PARK2, and the NSCLC has a deleterious mutation in RB1. In certain embodiments, the cancer is NSCLC, the NSCLC has a deleterious mutation in PARK2, and the NSCLC has a deleterious mutation in TP53. In certain embodiments, the cancer is NSCLC, and the NSCLC has a deleterious mutation in FBXW7. In certain embodiments, the cancer is NSCLC, the NSCLC has a deleterious mutation in FBXW7, and the NSCLC has an amplification or overexpression of MYC. In certain embodiments, the cancer is NSCLC, the NSCLC has a deleterious mutation in FBXW7, and the NSCLC has a deleterious mutation or loss of function mutation in CDKN2A. In certain embodiments, the cancer is NSCLC, the NSCLC has a deleterious mutation in FBXW7, and the NSCLC has a deleterious mutation or loss of function mutation in CDKN2B. In certain embodiments, the cancer is NSCLC, and the NSCLC has a deleterious mutation in FBXW7 and TP53. In certain embodiments, the cancer is NSCLC, the NSCLC has a deleterious mutation in FBXW7 and TP53, and the NSCLC has an amplification or overexpression of MYC. In certain embodiments, the cancer is NSCLC, the NSCLC has a deleterious mutation or loss of function mutation in CDKN2A and CDKN2B, and the NSCLC has a deleterious mutation in TP53 and FBXW7. In certain embodiments, the cancer is NSCLC, and the NSCLC has an amplification or overexpression of MYC. In certain embodiments, the cancer is NSCLC, the NSCLC has an amplification or overexpression of MYC, and the NSCLC has a deleterious mutation or loss of function mutation in CDKN2A. In certain embodiments, the cancer is NSCLC, the NSCLC has an amplification or overexpression of MYC, and the NSCLC has a deleterious mutation or loss of function mutation in CDKN2B. In certain embodiments, the cancer is NSCLC, the NSCLC has an amplification or overexpression of MYC, and the NSCLC has a deleterious mutation or loss of function mutation in CDKN2A and CDKN2B. In certain embodiments, the cancer is NSCLC, the NSCLC has an amplification or overexpression of MYC, and the NSCLC has a deleterious mutation in TP53. In certain embodiments, the cancer is NSCLC, the NSCLC has an amplification or overexpression of MYC, the NSCLC has a deleterious mutation or loss of function mutation in CDKN2A and CDKN2B, and the NSCLC has a deleterious mutation in TP53. In certain embodiments, the cancer is NSCLC, and the NSCLC has an amplification or overexpression of MYCN. In certain embodiments, the cancer is NSCLC, the NSCLC has an amplification or overexpression of MYCN, and the NSCLC has a deleterious mutation in RB1. In certain embodiments, the cancer is NSCLC, the NSCLC has an amplification or overexpression of MYCN, and the NSCLC has a deleterious mutation in TP53. In certain embodiments, the cancer is NSCLC, and the NSCLC has an amplification or overexpression of MYCL. In certain embodiments, the cancer is NSCLC, the NSCLC has an amplification or overexpression of MYCL, and the NSCLC has a deleterious mutation in RB1. In certain embodiments, the cancer is NSCLC, the NSCLC has an amplification or overexpression of MYCL, and the NSCLC has a deleterious mutation in TP53. In certain embodiments, the cancer is NSCLC, and the NSCLC has a deleterious mutation or loss of function mutation in CDKN2A. In certain embodiments, the cancer is NSCLC, and the NSCLC has a deleterious mutation or loss of function mutation in CDKN2B. In certain embodiments, the cancer is NSCLC, and the NSCLC has a deleterious mutation or loss of function mutation in CDKN2A and CDKN2B. In certain embodiments, the cancer is NSCLC, the NSCLC has a deleterious mutation or loss of function mutation in CDKN2A, and the NSCLC has a deleterious mutation in TP53. In certain embodiments, the cancer is NSCLC, the NSCLC has a deleterious mutation or loss of function mutation in CDKN2B, and the NSCLC has a deleterious mutation in TP53. In certain embodiments, the cancer is NSCLC, the NSCLC has a deleterious mutation or loss of function mutation in CDKN2A and CDKN2B, and the NSCLC has a deleterious mutation in TP53. In certain embodiments, the cancer is NSCLC, and the NSCLC has a deleterious mutation in RB1. In certain embodiments, the cancer is NSCLC, and the NSCLC has a deleterious mutation in RB1 and TP53. In certain embodiments, the cancer is NSCLC, and the NSCLC has a deleterious mutation in TP53.

In certain aspects, the present disclosure provides for methods of treating cancer, wherein the cancer is bladder cancer. In certain embodiments, the cancer is bladder cancer, and the bladder cancer has an amplification or overexpression of CCNE1. In certain embodiments, the cancer is bladder cancer, the bladder cancer has an amplification or overexpression of CCNE1, and the bladder cancer has a deleterious mutation in TP53. In certain embodiments, the cancer is bladder cancer, and the bladder cancer has a deleterious mutation in FBXW7. In certain embodiments, the cancer is bladder cancer, and the bladder cancer has a deleterious mutation in FBXW7 and TP53. In certain embodiments, the cancer is bladder cancer, the bladder cancer has a deleterious mutation in FBXW7, and the bladder cancer has a deleterious mutation or loss of function mutation in CDKN2A. In certain embodiments, the cancer is bladder cancer, the bladder cancer has a deleterious mutation in FBXW7, and the bladder cancer has a deleterious mutation or loss of function mutation in CDKN2B. In certain embodiments, the cancer is bladder cancer, and the bladder cancer has a deleterious mutation in PARK2. In certain embodiments, the cancer is bladder cancer, and the bladder cancer has a deleterious mutation in PARK2 and TP53. In certain embodiments, the cancer is bladder cancer, the bladder cancer has a deleterious mutation in PARK2, and the bladder cancer has a deleterious mutation or loss of function mutation in CDKN2A. In certain embodiments, the cancer is bladder cancer, the bladder cancer has a deleterious mutation in PARK2, and the bladder cancer has a deleterious mutation or loss of function mutation in CDKN2B. In certain embodiments, the cancer is bladder cancer, and the bladder cancer has an amplification or overexpression of MYC. In certain embodiments, the cancer is bladder cancer, the bladder cancer has an amplification or overexpression of MYC, and the bladder cancer has a deleterious mutation in TP53. In certain embodiments, the cancer is bladder cancer, the bladder cancer has an amplification or overexpression of MYC, and the bladder cancer has a deleterious mutation in RB1. In certain embodiments, the cancer is bladder cancer, and the bladder cancer has an amplification or overexpression of MYCN. In certain embodiments, the cancer is bladder cancer, the bladder cancer has an amplification or overexpression of MYCN, and the bladder cancer has a deleterious mutation in TP53. In certain embodiments, the cancer is bladder cancer, and the bladder cancer has an amplification or overexpression of MYCL. In certain embodiments, the cancer is bladder cancer, the bladder cancer has an amplification or overexpression of MYCL, and the bladder cancer has a deleterious mutation in TP53. In certain embodiments, the cancer is bladder cancer, the bladder cancer has an amplification or overexpression of MYCL, and the bladder cancer has a deleterious mutation or loss of function mutation in CDKN2A. In certain embodiments, the cancer is bladder cancer, the bladder cancer has an amplification or overexpression of MYCL, and the bladder cancer has a deleterious mutation or loss of function mutation in CDKN2B. In certain embodiments, the cancer is bladder cancer, and the bladder cancer has an amplification or overexpression of PIK3CA. In certain embodiments, the cancer is bladder cancer, the bladder cancer has an amplification or overexpression of PI3KCA, and the bladder cancer has a deleterious mutation in TP53. In certain embodiments, the cancer is bladder cancer, the bladder cancer has an amplification or overexpression of PI3KCA, and the bladder cancer has a deleterious mutation or loss of function mutation in CDKN2A. In certain embodiments, the cancer is bladder cancer, the bladder cancer has an amplification or overexpression of PI3KCA, and the bladder cancer has a deleterious mutation or loss of function mutation in CDKN2B. In certain embodiments, the cancer is bladder cancer, the bladder cancer has an amplification or overexpression of PI3KCA, and the bladder cancer has a deleterious mutation or loss of function mutation in CDKN1A. In certain embodiments, the cancer is bladder cancer, the bladder cancer has an amplification or overexpression of PI3KCA, and the bladder cancer has a mutation in MDM2. In certain embodiments, the cancer is bladder cancer, and the bladder cancer has a deleterious mutation or loss of function mutation in ATM. In certain embodiments, the cancer is bladder cancer, the bladder cancer has a deleterious mutation or loss of function mutation in ATM, and the bladder cancer has a deleterious mutation in TP53. In certain embodiments, the cancer is bladder cancer, and the bladder cancer has a deleterious mutation or loss of function mutation in ATM and CDKN2A. In certain embodiments, the cancer is bladder cancer, and the bladder cancer has a deleterious mutation or loss of function mutation in ATM and CDKN2B. In certain embodiments, the cancer is bladder cancer, the bladder cancer has a deleterious mutation or loss of function mutation in ATM, and the bladder cancer has a deleterious mutation in MLL2. In certain embodiments, the cancer is bladder cancer, and the bladder cancer has a deleterious mutation or loss of function mutation in BRCA1. In certain embodiments, the cancer is bladder cancer, the bladder cancer has a deleterious mutation or loss of function mutation in BRCA1, and the bladder cancer has a deleterious mutation in TP53. In certain embodiments, the cancer is bladder cancer, and the bladder cancer has a deleterious mutation or loss of function mutation in BRCA2. In certain embodiments, the cancer is bladder cancer, the bladder cancer has a deleterious mutation or loss of function mutation in BRCA2, and the bladder cancer has a deleterious mutation in TP53. In certain embodiments, the cancer is bladder cancer, and the bladder cancer has a deleterious mutation or loss of function mutation in BRCA2 and CDKN2A. In certain embodiments, the cancer is bladder cancer, and the bladder cancer has a deleterious mutation or loss of function mutation in BRCA2 and CDKN2B. In certain embodiments, the cancer is bladder cancer, the bladder cancer has a deleterious mutation or loss of function mutation in BRCA2, and the bladder cancer has a deleterious mutation in RB1. In certain embodiments, the cancer is bladder cancer, the bladder cancer has a deleterious mutation or loss of function mutation in BRCA2, and the bladder cancer has a deleterious mutation in ARID1A. In certain embodiments, the cancer is bladder cancer, and the bladder cancer has a deleterious mutation in ARID1A. In certain embodiments, the cancer is bladder cancer, and the bladder cancer has a deleterious mutation in ARID1A and TP53. In certain embodiments, the cancer is bladder cancer, the bladder cancer has a deleterious mutation or loss of function mutation in CDKN2A, and the bladder cancer has a deleterious mutation in ARID1A. In certain embodiments, the cancer is bladder cancer, and the bladder cancer has a deleterious mutation or loss of function mutation in ARID1A and CDKN2B. In certain embodiments, the cancer is bladder cancer, and the bladder cancer has a deleterious mutation in ARID1A and MLL2. In certain embodiments, the cancer is bladder cancer, the bladder cancer has a deleterious mutation in ARID1A, and the bladder cancer has an amplification or overexpression of PI3KCA. In certain embodiments, the cancer is bladder cancer, and the bladder cancer has a deleterious mutation in MLL2. In certain embodiments, the cancer is bladder cancer, and the bladder cancer has a deleterious mutation in MLL2 and TP53. In certain embodiments, the cancer is bladder cancer, and the bladder cancer has a deleterious mutation or loss of function mutation in MLL2 and CDKN2A. In certain embodiments, the cancer is bladder cancer, and the bladder cancer has a deleterious mutation or loss of function mutation in CDKN2B and a deleterious mutation in MLL2. In certain embodiments, the cancer is bladder cancer, and the bladder cancer has a deleterious mutation in MLL2 and RB1. In certain embodiments, the cancer is bladder cancer, and the bladder cancer has a deleterious mutation or loss of function mutation in CDKN1A. In certain embodiments, the cancer is bladder cancer, the bladder cancer has a deleterious mutation or loss of function mutation in CDKN1A, and the bladder cancer has a deleterious mutation in TP53. In certain embodiments, the cancer is bladder cancer, and the bladder cancer has a deleterious mutation or loss of function mutation in CDKN1A and CDKN2A. In certain embodiments, the cancer is bladder cancer, and the bladder cancer has a deleterious mutation or loss of function mutation in CDKN1A and CDKN2B. In certain embodiments, the cancer is bladder cancer, the bladder cancer has a deleterious mutation or loss of function mutation in CDKN1A, and the bladder cancer has a deleterious mutation in RB1. In certain embodiments, the cancer is bladder cancer, and the bladder cancer has a deleterious mutation or loss of function mutation in CDKN2A. In certain embodiments, the cancer is bladder cancer, the bladder cancer has a deleterious mutation or loss of function mutation in CDKN2A, and the bladder cancer has a deleterious mutation in TP53. In certain embodiments, the cancer is bladder cancer, and the bladder cancer has a deleterious mutation or loss of function mutation in CDKN2A and CDKN2B. In certain embodiments, the cancer is bladder cancer, the bladder cancer has a deleterious mutation or loss of function mutation in CDKN2A, and the bladder cancer has a deleterious mutation in MDM2. In certain embodiments, the cancer is bladder cancer, the bladder cancer has a deleterious mutation or loss of function mutation in CDKN2A, and the bladder cancer has a deleterious mutation in PTEN. In certain embodiments, the cancer is bladder cancer, the bladder cancer has a deleterious mutation or loss of function mutation in CDKN2A, and the bladder cancer has a deleterious mutation in RB1. In certain embodiments, the cancer is bladder cancer, and the bladder cancer has a deleterious mutation or loss of function mutation in CDKN2B. In certain embodiments, the cancer is bladder cancer, the bladder cancer has a deleterious mutation or loss of function mutation in CDKN2B, and the bladder cancer has a deleterious mutation in TP53. In certain embodiments, the cancer is bladder cancer, the bladder cancer has a deleterious mutation or loss of function mutation in CDKN2B, and the bladder cancer has a deleterious mutation in MDM2. In certain embodiments, the cancer is bladder cancer, the bladder cancer has a deleterious mutation or loss of function mutation in CDKN2B, and the bladder cancer has a deleterious mutation in PTEN. In certain embodiments, the cancer is bladder cancer, the bladder cancer has a deleterious mutation or loss of function mutation in CDKN2B, and the bladder cancer has a deleterious mutation in RB1. In certain embodiments, the cancer is bladder cancer, and the bladder cancer has a mutation in MDM2. In certain embodiments, the cancer is bladder cancer, and the bladder cancer has a deleterious mutation in RB1 and a mutation in MDM2. In certain embodiments, the cancer is bladder cancer, and the bladder cancer has a deleterious mutation in PTEN. In certain embodiments, the cancer is bladder cancer, and the bladder cancer has a deleterious mutation in PTEN and TP53. In certain embodiments, the cancer is bladder cancer, and the bladder cancer has a deleterious mutation in PTEN and RB1. In certain embodiments, the cancer is bladder cancer, and the bladder cancer has a deleterious mutation in RB1. In certain embodiments, the cancer is bladder cancer, and the bladder cancer has a deleterious mutation in RB1 and TP53. In certain embodiments, the cancer is bladder cancer, and the bladder cancer has a deleterious mutation in TP53.

In certain aspects, the present disclosure provides for methods of treating cancer, wherein the cancer is small cell lung cancer (SCLC). In certain embodiments, the cancer is SCLC, and the SCLC has a deleterious mutation in FBXW7. In certain embodiments, the cancer is SCLC, and the SCLC has a deleterious mutation in FBXW7 and TP53. In certain embodiments, the cancer is SCLC, and the SCLC has a deleterious mutation in FBXW7 and RB1. In certain embodiments, the cancer is SCLC, the SCLC has a deleterious mutation in FBXW7, and the SCLC has a deleterious mutation in RB1 and TP53. In certain embodiments, the cancer is SCLC, and the SCLC has a deleterious mutation in MLL2. In certain embodiments, the cancer is SCLC, and the SCLC has a deleterious mutation in MLL2 and TP53. In certain embodiments, the cancer is SCLC, and the SCLC has a deleterious mutation in MLL2 and RB1. In certain embodiments, the cancer is SCLC, the SCLC has a deleterious mutation in MLL2, RB1 and TP53. In certain embodiments, the cancer is SCLC, and the SCLC has a deleterious mutation or loss of function mutation in CDKN2A. In certain embodiments, the cancer is SCLC, and the SCLC has a deleterious mutation or loss of function mutation in CDKN2A, and the SCLC has a deleterious mutation in MLL2 and TP53. In certain embodiments, the cancer is SCLC, the SCLC has a deleterious mutation or loss of function mutation in CDKN2A, and the SCLC has a deleterious mutation in RB1. In certain embodiments, the cancer is SCLC, the SCLC has a deleterious mutation or loss of function mutation in CDKN2A, and the SCLC has a deleterious mutation in RB1 and TP53. In certain embodiments, the cancer is SCLC, and the SCLC has a deleterious mutation in PTEN. In certain embodiments, the cancer is SCLC, and the SCLC has a deleterious mutation in PTEN and TP53. In certain embodiments, the cancer is SCLC, and the SCLC has a deleterious mutation in PTEN and RB1. In certain embodiments, the cancer is SCLC, and the SCLC has a deleterious mutation in PTEN, RB1 and TP53. In certain embodiments, the cancer is SCLC, and the SCLC has a deleterious mutation in RB1. In certain embodiments, the cancer is SCLC, and the SCLC has a deleterious mutation in RB1 and TP53. In certain embodiments, the cancer is SCLC, and the SCLC has a deleterious mutation in TP53.

In certain aspects, the present disclosure provides for methods of treating cancer, wherein the cancer is cervical cancer. In certain embodiments, the cancer is cervical cancer, and the cervical cancer has an amplification or overexpression in CCNE1. In certain embodiments, the cancer is cervical cancer, the cervical cancer has an amplification or overexpression in CCNE1, and the cervical cancer is positive for HPV. In certain embodiments, the cancer is cervical cancer, the cervical cancer has an amplification or overexpression in CCNE1, and the cervical cancer has a deleterious mutation in TP53. In certain embodiments, the cancer is cervical cancer, and the cervical cancer has a deleterious mutation in FBXW7. In certain embodiments, the cancer is cervical cancer, the cervical cancer has a deleterious mutation in FBXW7, and the cervical cancer is positive for HPV. In certain embodiments, the cancer is cervical cancer, and the cervical cancer has a deleterious mutation in FBXW7 and TP53. In certain embodiments, the cancer is cervical cancer, and the cervical cancer has a deleterious mutation in PARK2. In certain embodiments, the cancer is cervical cancer, the cervical cancer has a deleterious mutation in PARK2, and the cervical cancer is positive for HPV. In certain embodiments, the cancer is cervical cancer, the cervical cancer has a deleterious mutation in PARK2, and the cervical cancer has a deleterious mutation or loss of function mutation in ATM. In certain embodiments, the cancer is cervical cancer, and the cervical cancer has an amplification or overexpression in MYC. In certain embodiments, the cancer is cervical cancer, the cervical cancer has an amplification or overexpression in MYC, and the cervical cancer is positive for HPV. In certain embodiments, the cancer is cervical cancer, and the cervical cancer has an amplification or overexpression in MYCN. In certain embodiments, the cancer is cervical cancer, the cervical cancer has an amplification or overexpression in MYCN, and the cervical cancer is positive for HPV. In certain embodiments, the cancer is cervical cancer, and the cervical cancer has an amplification or overexpression in MYCL. In certain embodiments, the cancer is cervical cancer, the cervical cancer has an amplification or overexpression in MYCL, and the cervical cancer is positive for HPV. In certain embodiments, the cancer is cervical cancer, and the cervical cancer has a deleterious mutation or loss of function mutation in ATM. In certain embodiments, the cancer is cervical cancer, the cervical cancer has a deleterious mutation or loss of function mutation in ATM, and the cervical cancer is positive for HPV. In certain embodiments, the cancer is cervical cancer, and the cervical cancer has a deleterious mutation or loss of function mutation in BRCA1. In certain embodiments, the cancer is cervical cancer, the cervical cancer has a deleterious mutation or loss of function mutation in BRCA1, and the cervical cancer is positive for HPV. In certain embodiments, the cancer is cervical cancer, and the cervical cancer has a deleterious mutation or loss of function mutation in BRCA2. In certain embodiments, the cancer is cervical cancer, the cervical cancer has a deleterious mutation or loss of function mutation in BRCA2, and the cervical cancer is positive for HPV. In certain embodiments, the cancer is cervical cancer, the cervical cancer has a deleterious mutation or loss of function mutation in BRCA2, and the cervical cancer has a deleterious mutation in RB1. In certain embodiments, the cancer is cervical cancer, and the cervical cancer has a deleterious mutation in ARID1A. In certain embodiments, the cancer is cervical cancer, the cervical cancer has a deleterious mutation in ARID1A, and the cervical cancer is positive for HPV. In certain embodiments, the cancer is cervical cancer, the cervical cancer has a deleterious mutation in ARID1A, and the cervical cancer has an amplification or overexpression in PIK3CA. In certain embodiments, the cancer is cervical cancer, and the cervical cancer has a deleterious mutation in ARID1A and PTEN. In certain embodiments, the cancer is cervical cancer, and the cervical cancer has a deleterious mutation in ARID1A and RB1. In certain embodiments, the cancer is cervical cancer, and the cervical cancer has an amplification or overexpression in PIK3CA. In certain embodiments, the cancer is cervical cancer, the cervical cancer has an amplification or overexpression in PIK3CA, and the cervical cancer is positive for HPV. In certain embodiments, the cancer is cervical cancer, and the cervical cancer has a deleterious mutation in PTEN. In certain embodiments, the cancer is cervical cancer, the cervical cancer has a deleterious mutation in PTEN, and the cervical cancer is positive for HPV. In certain embodiments, the cancer is cervical cancer, and the cervical cancer has a deleterious mutation in PTEN and RB1. In certain embodiments, the cancer is cervical cancer, and the cervical cancer has a deleterious mutation in RB1. In certain embodiments, the cancer is cervical cancer, the cervical cancer has a deleterious mutation in RB1, and the cervical cancer is positive for HPV. In certain embodiments, the cancer is cervical cancer, and the cervical cancer has a deleterious mutation in RB1 and TP53. In certain embodiments, the cancer is cervical cancer, and the cervical cancer has a deleterious mutation in STK11. In certain embodiments, the cancer is cervical cancer, the cervical cancer has a deleterious mutation in STK11, and the cervical cancer is positive for HPV. In certain embodiments, the cancer is cervical cancer, and the cervical cancer is positive for HPV. In certain embodiments, the cancer is cervical cancer, and the cervical cancer has a deleterious mutation in TP53.

In certain aspects, the present disclosure provides for methods of treating cancer, wherein the cancer is sarcoma. In certain embodiments, the cancer is sarcoma, and the sarcoma has a deleterious mutation in TP53. In certain embodiments, the cancer is sarcoma, the sarcoma has a deleterious mutation in TP53, and the sarcoma has a mutation in MDM2. In certain embodiments, the cancer is sarcoma, and the sarcoma has an amplification or overexpression in CCNE1. In certain embodiments, the cancer is sarcoma, the sarcoma has an amplification or overexpression in CCNE1, and the sarcoma has a deleterious mutation in FBXW7. In certain embodiments, the cancer is sarcoma, the sarcoma has an amplification or overexpression in CCNE1, and the sarcoma has a deleterious mutation in PARK2. In certain embodiments, the cancer is sarcoma, and the sarcoma has a deleterious mutation or loss of function mutation in CDKN2A. In certain embodiments, the cancer is sarcoma, and the sarcoma has a deleterious mutation or loss of function mutation in CDKN2B. In certain embodiments, the cancer is sarcoma, and the sarcoma has a deleterious mutation or loss of function mutation in CDKN2A and CDKN2B. In certain embodiments, the cancer is sarcoma, and the sarcoma has a deleterious mutation in RB1. In certain embodiments, the cancer is sarcoma, and the sarcoma has an amplification or overexpression in MYC. In certain embodiments, the cancer is sarcoma, and the sarcoma has an amplification or overexpression in MYCN. In certain embodiments, the cancer is sarcoma, and the sarcoma has a deleterious mutation in PTEN. In certain embodiments, the cancer is sarcoma, and the sarcoma has an amplification or overexpression in PIK3CA. In certain embodiments, the cancer is sarcoma, and the sarcoma has a deleterious mutation or loss of function mutation in a DDR pathway gene. In certain embodiments, the cancer is sarcoma, and the sarcoma has a deleterious mutation or loss of function mutation in ATM. In certain embodiments, the cancer is sarcoma, and the sarcoma has a deleterious mutation or loss of function mutation in BRCA1. In certain embodiments, the cancer is sarcoma, and the sarcoma has a deleterious mutation or loss of function mutation in BRCA2. In certain embodiments, the cancer is sarcoma, and the sarcoma has an amplification, overexpression or gain of function mutation in CHEK1. In certain embodiments, the cancer is sarcoma, and the sarcoma has an amplification, overexpression or gain of function mutation in ATR.

The cancer types with genetic alterations listed above are examples of non-limiting embodiments for the methods of the invention. For any of the above described types of cancers, the cancers can harbor any number of additional genetic alterations.

### Dosing Regimens

The methods disclosed herein comprise administration of a Chk1 inhibitor. In certain embodiments, the Chk1 inhibitor is SRA737. In certain embodiments the Chk1 inhibitor is administered as a monotherapy or in combination with other anti-cancer agents (e.g., chemotherapeutic agents). In certain embodiments, SRA737 is administered orally (PO), at a dose of 1-300 mg/kg/day. In certain embodiments, SRA737 is administered at a dose of 1-100 mg/kg/day PO. In certain embodiments, SRA737 is administered orally (PO), at a dose of 1-50 mg/kg/day. In certain embodiments, SRA737 is administered orally (PO), at a dose of 1.5-35 mg/kg/day. In certain embodiments, SRA737 is administered at a dose of 1-300 mg/kg/day PO, daily for at least one cycle, wherein a cycle is 1-35 days. In certain embodiments, SRA737 is administered at a dose of 1-300 mg/kg/day PO, daily for 5 days. In certain embodiments, SRA737 is administered at a dose of 1-300 mg/kg/day PO, daily for 5 days followed by 2 days with no administration of SRA 737. In certain embodiments, SRA737 is administered at a dose of 1.7-33.3 mg/kg/day PO, daily for 5 days followed by 2 days with no administration of SRA 737. Dosing schedules may include, but are not limited to, the following examples: daily dosing, 5 days of dosing followed by 2 days of non-dosing each week; 1 week of daily dosing followed by 1, 2, or 3 weeks of non-dosing; 2 or 3 weeks of daily dosing followed by 1, or 2 weeks of non-dosing; or twice daily dosing.

In certain embodiments the methods disclosed herein comprise administration of a Chk1 inhibitor in combination with a genotoxic agent. In certain embodiments, the methods comprise administration of SRA737 in combination with gemcitabine. In certain embodiments, SRA737 is administered at a dose of 1-300 mg/kg/day PO, for at least one day for at least one cycle, wherein the cycle is 1-35 days, and gemcitabine is administered at a dose of 1-200 mg/kg intravenously (IV) for at least one day for at least one cycle. In certain embodiments, SRA737 is administered at a dose of 1-300 mg/kg/day PO, for at least one day for at least one cycle, wherein the cycle is 28 days, Dosing schedules for either SRA 737 and/or gemcitabine may include, but are not limited to, the following examples: daily dosing, 5 days of dosing followed by 2 days of non-dosing each week; 1 week of daily dosing followed by 1, 2, or 3 weeks of non-dosing; 2 or 3 weeks of daily dosing followed by 1, or 2 weeks of non-dosing; or twice daily dosing.

### Compounds of the Invention

In certain embodiments, the present disclosure provides for methods of use of the Chk1 inhibitor, SRA737. The compound SRA737 is also identified by the chemical name: 5-[[4-[[morpholin-2-yl]methylamino]-5-(trifluoromethyl)-2-pyridyl]amino]pyrazine-2-carbonitrile. Each of the enantiomers of SRA737 is useful for compositions and methods disclosed herein.

SRA737 is a compound that is disclosed in international patent application no. PCT/GB2013/051233, which is herein incorporated by reference. The skilled artisan will find the how to synthesize SRA737 in international patent application no. PCT/GB2013/051233.

In one aspect, the SRA737 structures are as shown in the table below.

| Description | Structure |
|---|---|
| SRA737 structure | 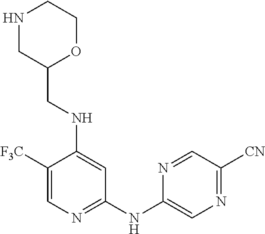 [SMILES: N#Cc1cnc(Nc2cc(NCC3CNCCO3)c(C(F)(F)F)cn2)cn1] |

### Pharmaceutical Compositions of the Invention

Methods for inhibiting the growth of a tumor, inhibiting the progression of or treating cancer are described herein. In certain embodiments, the methods of the invention include administering a therapeutically effective amount of a Chk1 inhibitor. In certain embodiments, the methods of the invention include administering a therapeutically effective amount of a Chk1 inhibitor and a genotoxic agent. The active compounds of the invention can each be formulated in pharmaceutical compositions. These pharmaceutical compositions may comprise, in addition to the active compound(s), a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

A composition can be administered alone or in combination with other treatments, either simultaneously or sequentially dependent upon the condition to be treated.

In general, the compounds of the present technology will be administered in a therapeutically effective amount by any of the accepted modes of administration for agents that serve similar utilities. The actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the present technology, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is therapeutic index, which can be expressed as the ratio LD₅₀/ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present technology is not limited to any particular composition or pharmaceutical carrier, as such may vary. In general, compounds of the present technology will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the present technology is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes therapeutic agents (which are formulated in a liquid form) to spray as a mist that is carried into the subject's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the subject's inspiratory air-stream during breathing by the device. In order to achieve a free flowing powder, therapeutic agent is formulated with an excipient such as lactose. A measured amount of therapeutic agent is stored in a capsule form and is dispensed with each actuation.

Pharmaceutical dosage forms of a compound of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present technology can include one or more physiologically acceptable inactive ingredients that facilitate processing of active molecules into preparations for pharmaceutical use.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present technology in combination with at least one pharmaceutically acceptable excipient. Acceptable excipients are non-toxic, aid administration, and do not adversely affect therapeutic benefit of the claimed compounds. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the present technology in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

In some embodiments, the pharmaceutical compositions include a pharmaceutically acceptable salt. The term “pharmaceutically acceptable salt” refers to salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in Stahl and Wermuth (Eds.), Handbook of Pharmaceutical Salts Properties, Selection, and Use; 2002.

The present compositions may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compositions comprising a compound of the present technology formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of the present technology based on the total formulation, with the balance being one or more suitable pharmaceutical excipients. Preferably, the compound is present at a level of about 1-80 wt %.

Advantages and Utility

Briefly, and as described in more detail below, described herein are improved methods for the treatment of cancer comprising administration of inhibitors of Chk1. In addition, the invention provides for novel genetically-based prospective patient enrichment strategies for the use of cancer treatment regimens comprising inhibitors of Chk1.

### Definitions

Terms used in the claims and specification are defined as set forth below unless otherwise specified.

The term “sufficient amount” means an amount sufficient to produce a desired effect, e.g., an amount sufficient to modulate Chk1 in a cell, or an amount sufficient to reduce tumor growth in a patient.

The term “therapeutically effective amount” is an amount that is effective to ameliorate a symptom of a disease. A therapeutically effective amount can be a “prophylactically effective amount” as prophylaxis can be considered therapy.

The term “administering” or “administration” of a drug and/or therapy to a subject (and grammatical equivalents of this phrase) refers to both direct or indirect administration, which may be administration to a subject by a medical professional, may be self-administration, and/or indirect administration, which may be the act of prescribing or inducing one to prescribe a drug and/or therapy to a subject.

The term “treating” or “treatment of” a disorder or disease refers to taking steps to alleviate the symptoms of the disorder or disease, or otherwise obtain some beneficial or desired results for a subject, including clinical results. Any beneficial or desired clinical results may include, but are not limited to, prevention, alleviation or amelioration of one or more symptoms of cancer or conditional survival and reduction of tumor load or tumor volume; diminishment of the extent of the disease; delay or slowing of the tumor progression or disease progression; amelioration, palliation, or stabilization of the tumor and/or the disease state; or other beneficial results.

The term “in situ” or “in vitro” refers to processes that occur in a living cell growing separate from a living organism, e.g., growing in tissue culture.

The term “in vivo” refers to processes that occur in a living organism.

The term “genetic alterations” refers to any change in the genome leading to a change in DNA sequence, mRNA sequence, protein sequence, changes in gene expression (either mRNA or protein abundance), or combinations thereof. Genetic alterations includes deleterious mutations (e.g., mutations that reduce or abolish either gene function or gene expression), loss of function mutations, and gain of function mutations. Genetic alterations includes insertions of viral genetic material into the genome of infected host cells (e.g., human papillomavirus). Genetic alterations also includes microsatellites or other repetitive tracts of DNA (e.g., short tandem repeats or simple sequence repeats).

The term “overexpression” when referring to a gene (e.g., an oncogenic driver gene), refers to any increase in mRNA, protein, or combinations thereof corresponding to a gene compared to at least one reference sample.

The term “microsatellite instability” refers to tumors that are characterized by having repetitive DNA sequences (e.g., short tandem repeats or simple sequence repeats). In certain embodiments, microsatellite instability is characterized by detection of mononucleotide repeat markers (e.g., BAT-25, BAT-26, NR-21, NR-24 and MONO-27). Methods for detection of microsatellite instability include any methods known in the art including, but not limited to, those methods described in Wang M. et al., Screening for Microsatellite Instability in Colorectal Cancer and Lynch Syndrome—A Mini Review; N A J Med Sci. 2016; 9(1):5-11. The term “microsatellite instability” in certain embodiments also refers to tumors that are characterized by having one or more repetitive DNA sequences known in the art to be correlated with loss of mismatch repair (MMR) compared to at least one reference sample.

The term “Chk1” or “CHEK1” or “checkpoint kinase 1” refers to serine/threonine-protein kinase that is encoded by the CHEK1 gene. CHEK1 can also be referred to as Cell Cycle Checkpoint Kinase, CHK1 Checkpoint Homolog, EC 2.7.11.1 and EC 2.7.11. Chk1 refers to all alternatively spliced analogues and comprises Homo sapiens Chk1 isoforms encoded by amino acid sequences and nucleotide sequences according to National Center for Biotechnology Information (NCBI) accession numbers: NP_001107594.1, NP_001107593.1, NP_001265.2, NP_001231775.1, NP_001317356.1, NP_001317357.1, XP_016872635.1, XP_024304105.1, and XP_011540862.1, NM_001114122, NM_001114121.2, NM_001274.5, NM_001244846.1, NM_001330427.1, NM_001330428.1, and XM_017017146.2.

The term “Chk1 inhibitor” refers to and inhibitor of Chk1 or CHEK1. A Chk1 inhibitor may be a small molecule, an antibody or a nucleic acid. A Chk1 inhibitor may reduce the expression of CHEK1, inhibit the activity or function of Chk1 in cells, or combinations thereof. Chk1 inhibitors include, but are not limited to: SRA737, Prexasertib (LY2606368) (Commercially available from Sellechchem, Catalog No. S7178), PF-477736 (Commercially available from Sellechchem, Catalog No. S2904), AZD7762 (Commercially available from Sellechchem, Catalog No. S1532), Rabusertib (LY2603618) (Commercially available from Sellechchem, Catalog No. S2626), MK-8776 (SCH 900776) (Commercially available from Sellechchem, Catalog No. S2735), CHIR-124 (Commercially available from Sellechchem, Catalog No. S2683), SAR-020106 (Commercially available from Sellechchem, Catalog No. 57740) and CCT245737 (Commercially available from Sellechchem, Catalog No. S8253).

The term “ATR inhibitor” or “inhibitor of ATR” refers to any inhibitor of ATR, and any alternatively spliced analogues. An ATR inhibitor may be a small molecule, an antibody or a nucleic acid. An ATR inhibitor may reduce the expression of ATR, inhibit the activity or function of ATR in cells, or combinations thereof.

The term “PARP” refers to poly ADP-ribose polymerase. The term “PARP” refers to all members of the PARP family, including: PARP1, PARP2, VPARP (ParP4), Tankyrase-1 and -2 (PARP-5a or TNKS, and PARPa5b or TNKS2), PARP3, PARP6, TIPARP (or PARP7), PARP8, PARP9, PARP10, PARP11, PARP12, PARP14, PARP15, PARP16, and any alternatively spliced analogues.

The term “PARP inhibitor”, “inhibitor of PARP” or “PARPi” refers to an inhibitor of any PARP family member described above. A PARPi may be a small molecule, an antibody or a nucleic acid. A PARPi may reduce the expression of PARP, inhibit the activity or function of PARP in cells, or combinations thereof. PARPi include inhibitors that do or do not alter the binding of PARP to DNA. PARPi may inhibit any members of the PARP family. PARPi include, but are not limited to: Olaparib (AZD2281) (commercially available from Chemietek, catalog number CT-A2281, LC Laboratories®, catalog number 0-9201 and Selleckchem catalog number, S1060), Rucaparib (PF-01367338) (commercially available from Chemietek, catalog number CT-AG01, LC Laboratories® catalog number, R-6399 and Selleckchem, catalog number S1098), Veliparib (ABT-888) (commercially available from Chemietek, catalog number CT-A888, LC Laboratories®, catalog number V-4703 and Selleckchem, catalog number S1004), Niraparib (MK-4827) (commercially available from Chemietek, catalog number CT-MK4827, Selleckchem, catalog number S7625), Iniparib (BSI-201) (commercially available from Chemitek, catalog number CT-BSI201, Selleckchem, catalog number S1087), Talazoparib (BMN673) (commercially available from Selleckchem, catalog number S7048), 3-aminobenzamine (INO-1001) (commercially available from Selleckchem, catalog number S1132), Fluzoparib, BGB-290 (commercially available from MedKoo Biosciences, Inc. catalog number 206852), CEP-9722 (commercially available from MedKoo Biosciences, Inc. catalog number 204910), and SC-10914.

The term “reduction” of a symptom or symptoms (and grammatical equivalents of this phrase) refers to decreasing the severity or frequency of the symptom(s), or elimination of the symptom(s).

The term “tumor suppressor gene” refers to any gene that increases a “hallmark of tumor growth or cancer” when subjected to a deleterious mutation and/or is inhibited, deleted, reduced in expression, or otherwise has reduced function in a cell. “Hallmarks of tumor growth or cancer” include, but are not limited to, sustained or increased proliferation of a cell, sustained or increased proliferative signaling in a cell, replicative immortality, resisting cell death (e.g., apoptosis), evasion of growth suppression, avoidance of immune destruction, induction of angiogenesis, activation of invasive or metastatic potential, promotion of inflammation, deregulated cellular energetics, genome instability, and combinations thereof. Tumor suppressor genes include, but are not limited to, the following genes: RB1, TP53, ATM, RAD50, FBXW7, PARK2, CDKN2A, CDKN2B, PTEN, MLL2, ARID1A, STK11 and any alternatively spliced analogues. In addition, a gain of function mutations of certain genes may function as tumor suppressor genes, such as MDM2, and may confer sensitivity to Chk1 inhibition. RB1 can also be referred to as: RB, Retinoblastoma 1, Retinoblastoma-Associated Protein, RB Transcriptional Corepressor, Protein Phosphatase 1 Regulatory Subunit 130, P105-Rb, Pp110, and PRb. TP53 can also be referred to as: P53, Tumor Protein 53, Phosphoprotein P53, P53 Tumor Suppressor, Tumor Suppressor P53, TRP53, Antigen NY-CO-13, BCC7, and LFS1. ATM can also be referred to as: ATM Serine/Threonine Kinase, Ataxia Telangiectasia Mutated, A-T mutated, TELO1, TEL1, ATDC, AT1, ATE, ATA, ATC and ATD. RAD50 can also be referred to as: RAD50 Double Strand Break Repair Protein, HRad50, DNA Repair Protein RAD50, RAD502 and NBSLD. FBW7 can also be referred to as: F-Box and WD Repeat Domain Containing 7, F-Box and WD Repeat Domain Containing 7, E3 Ubiquitin Protein Ligase, F-BOX Protein FBX30, Fbx30, SEL-10, SEL10, HCdc4, FBW7, HAGO, Archipelago Homolog, F-Box Protein SEL-10, Archipelago, FBXO30, FBW6, CDC4, FBW6 and AGO. PARK2 can also be referred to as: Parkin RBR E3 Ubiquitin Protein Ligase, Parkinson Disease 2, Parkinson Protein 2 E3 Ubiquitin Protein Ligase, Parkinson Juvenile Disease Protein 2, Parkin E3 Ubiquitin Protein Ligase, Parkin, AR-JP, LPRS2 and PDJ. CDKN2A can also be referred to as: Cyclin Dependent Kinase Inhibitor 2A, Cyclin-Dependent Kinase 4 Inhibitor A, Multiple Tumor Suppressor 1, P16-INK4A, P14ARF, CDKN2, CDK4I, MTS-1, MTS1 and MLM. CDKN2B can also be referred to as: Cyclin Dependent Kinase Inhibitor 2B, Cyclin-Dependent Kinase 4 Inhibitor B, Multiple Tumor Suppressor 2, P14-INK4b, P15-INK4b, MTS-2, MTS2, P14 CDK Inhibitor, P15 CDK Inhibitor, CDK4B inhibitor, INK4B, TP15 and P15. PTEN can also be referred to as: Phosphatase and Tensin Homolog, MMAC1, TEP1, Phosphatidylinositol 3, 4, 5-Triphosphate 3-Phosphatase and Dual-Specificity Protein Phophatase, MMAC1 Phosphatase and Tensin Homolog Deleted on Chromosome 10, Mitochondrial Phosphatase and Tensin Protein Alpha, Phosphatase and Tensin-Like Protein, Mitochondrial PTEN alpha, PTEN1, CWS1, GLM2, MHAM, DEC and BZS. MLL2 can also be referred to as: Lysine Methyltransferase 2D, Myeloid/Lymphoid or Mixed-lineage Leukemia 2, Lysine (K)-Specific Methyltransferase 2D, Trinucleotide Repeat Containing 2D, Trinucleotide Repeat Containing 21, Lysine N-Methyltransferase 2D, ALL1-Related Protein, MLL4, ALR, Histone-Lysine N-Methyltransferase 2D, Kabuki Mental Retardation Syndrome, CAGL114, KABUK1, TNRC21, AAD10, and KMS. ARID1A can also be referred to as: AT-Rich Interaction Domain 1A, SWI/SNF-Related, Matrix-Associated, Actin-Dependent Regulator of Chromatin Subfamily F Member 1, AT Rich Interactive Domain 1A, ARID Domain-Containing Protein 1A, SWI/SNF Complex Protein P270, BRG1-Associated Factor 250a, SWI-Like Protein, Osa Homolog 1, BAF250a, SMARCF1, C1orf4, BAF250, HOSA1, B120, OSA1, HELD, SWI/SNF Related Matrix Associated Actin Dependent Regulator of Chromatin Subfamily F Member 1, AT-Rich Interactive Domain 1A, Chromatin Remodeling Factor P250, BRG-1 Associated Factor P250, BRG-1 Associated Factor 250, OSA1 Nuclear Protein, Brain Protein 120, BM029, MRD14, CSS2, P270 and ELD. STK11 can also be referred to as: Serine/Threonine Kinase 11, Polarization-Related Protein LKB1, PJS, Serine/Threonine Kinase 11 Peutz-Jeghers Syndrome, Serine/Threonine-Protein Kinase LKB1, Polarization-Related Protein LKB1, Renal Carcinoma Antigen NY-REN-19, Liver Kinase B1, HLKB1 and LKB1. MDM2 can also be referred to as: MDM2 Proto-Oncogene, MDM2 Proto-Oncogene E3 Ubiquitin Protein Ligase, Oncoprotein Mdm2, Hdm2, Mdm2 Transformed 3T3 Cell Double Minute 2 P53 Binding Protein, Double Minute 2 Human Homolog of P53-Binding Protein, RING-Type E3 Ubiquitin Transferase Mdm2, P53-Binding Protein Mdm2, Double Minute 2 Protein, ACTFS and HDMX. RB1 comprises Homo sapiens RB1 encoded by amino acid sequences and nucleotide sequences according to NCBI accession numbers: NP_000312 and NM_000321.2. TP53 comprises Homo sapiens TP53 encoded by amino acid sequences and nucleotide sequences according to NCBI accession numbers: NP_000537 and NM_000546.5. ATM comprises Homo sapiens ATM encoded by amino acid sequences and nucleotide sequences according to NCBI accession numbers: NP_000042 and NM_000051.3. RAD50 comprises Homo sapiens RAD50 encoded by amino acid sequences and nucleotide sequences according to NCBI accession numbers: NP_005723 and NM_005732.3. PARK2 comprises Homo sapiens PARK2 encoded by amino acid sequences and nucleotide sequences according to NCBI accession numbers: NM_004562 and NP_004553.2. CDKN2A comprises Homo sapiens CDKN2A encoded by amino acid sequences and nucleotide sequences according to NCBI accession numbers: NP_478102 and NM_058195.3. CDKN2B comprises Homo sapiens CDKN2B encoded by amino acid sequences and nucleotide sequences according to NCBI accession numbers: NP_004927 and NM_004936.3. PTEN comprises Homo sapiens PTEN encoded by amino acid sequences and nucleotide sequences according to NCBI accession numbers: NM_000314 and NP_000305.3. MLL2 comprises Homo sapiens MLL2 encoded by amino acid sequences and nucleotide sequences according to NCBI accession numbers: NM_003482 and NP_003473.3. ARID1A comprises Homo sapiens ARID1A encoded by amino acid sequences and nucleotide sequences according to NCBI accession numbers: NM_139135 and NP_624361.1. STK11 comprises Homo sapiens STK11 encoded by amino acid sequences and nucleotide sequences according to NCBI accession numbers: NM_000455 and NP_000446.1. MDM2 comprises Homo sapiens MDM2 encoded by amino acid sequences and nucleotide sequences according to NCBI accession numbers: NP_002383, NM_002392.5 and Q00987.

The term “DNA damage repair (DDR) gene” or “DNA damage repair pathway gene” refers to any gene that directly or indirectly promotes repair of DNA mutations, breaks or other DNA damage or structural changes. DNA damage repair genes include, but are not limited to, the following genes: ATM, BRCA1, BRCA2, MRE11A, ATR, POLE, MLH1, MSH3, Rad50, Rad51D, and any alternatively spliced analogues. ATM can also be referred to as: ATM Serine/Threonine Kinase, Ataxia Telangiectasia Mutated, A-T mutated, TELO1, TEL1, ATDC, AT1, ATE, ATA, ATC and ATD. BRCA1 is also referred to as: BRCA/BRCA1-Containing Complex Subunit 1, Protein Phosphatase 1 Regulatory Subunit 53, Fanconi Anemia Complementation Group S, RING Finger Protein 53, BROVCA1, PPP1R53, BRCAI and BRCC1. BRCA2 can also be referred to as: BRCA/BRCA1-Containing Complex Subunit 2, Fanconi Anemia Group D1 Protein, Fanconi Anemia complementation Group D1, Breast Cancer 2 Tumor Suppressor, Breast and Ovarian Cancer Susceptibility Protein 2, FANCD1, FACD, FANCD, FAD1, GLM3 and FAD. MRE11A can also be referred to as: MRE11 Homolog Double Strand Break Repair Nuclease, Meiotic Recombination 11 Homolog A, Meiotic recombination 11 Homolog 1, Double-Strand Break Repair Protein MRE11A, AT-Like Disease and MRE11 Homolog 1. ATR can also be referred to as: ATR Serine/Threonine Kinase, Ataxia Telangiectasia and RAD3-Related Protein, FRP1, MEC1 Mitosis Entry Checkpoint 1, FRAP Related Protein 1, FCTCS, SCKL1, MEC1 and SCKL. POLE can also be referred to as: DNA Polymerase Epsilon Catalytic Subunit, DNA Polymerase Epsilon Catalytic Subunit A, DNA Polymerase II Subunit A, POLE1, CRCS12 and FILS. MLH1 can also be referred to as: MutL Homolog 1, DNA Mismatch Repair Protein Mlh1, COCA2, HNPCC, HNPCC, HMLH1 and FCC2. MSH3 can be referred to as: DNA Mismatch Repair Protein MSH3, MutS Homolog 3, Divergent Upstream Protein, Mismatch Repair Protein1, HMSH3, MRP1, DNA Mismatch Repair Protein Msh3, FAP4, DUC1 and DUG. RAD50 can also be referred to as: RAD50 Double Strand Break Repair Protein, HRad50, DNA Repair Protein RAD50, RAD502 and NBSLD. RAD51D can also be referred to as: RAD51 Paralog D, RAD51 Homolog 4, RAD51-Like Protein 3, RAD51L3, R51H3, DNA Repair Protein RAD51 Homolog 4, TRAD and BROVCA4. ATM comprises Homo sapiens ATM encoded by amino acid sequences and nucleotide sequences according to NCBI accession numbers: NP_000042 and NM_000051.3. BRCA1 comprises Homo sapiens BRCA1 encoded by amino acid sequences and nucleotide sequences according to NCBI accession numbers: NP_009225 and NM_007294.3. BRCA2 comprises Homo sapiens BRCA2 encoded by amino acid sequences and nucleotide sequences according to NCBI accession numbers: NP_000050 and NM_000059.3. MRE11A comprises Homo sapiens MRE11A encoded by amino acid sequences and nucleotide sequences according to NCBI accession numbers: NM_005591 and NP_005582.1. ATR comprises Homo sapiens ATR encoded by amino acid sequences and nucleotide sequences according to NCBI accession numbers: NP_001175 and NM_001184.3. POLE comprises Homo sapiens POLE encoded by amino acid sequences and nucleotide sequences according to NCBI accession numbers: NP_006222 and NM_006231.3. MLH1 comprises Homo sapiens MLH1 encoded by amino acid sequences and nucleotide sequences according to NCBI accession numbers: NP_000240 and NM_000249.3. MSH3 comprises Homo sapiens MSH3 encoded by amino acid sequences and nucleotide sequences according to NCBI accession numbers: NM_002439 and NP_002430.3. RAD50 comprises Homo sapiens RAD50 encoded by amino acid sequences and nucleotide sequences according to NCBI accession numbers: NP_005723 and NM_005732.3. RAD51D comprises Homo sapiens RAD51D encoded by amino acid sequences and nucleotide sequences according to NCBI accession numbers: NP_002869 and NM_002878.3.

DDR genes also include genes in the Fanconi anemia (FA) pathway. Genes in the FA pathway include, but are not limited to, Fanconi anemia complementation group (FANC) genes, such as FANCA, FANCC, FANCD2, FANCE, FANCF, FANCG, FANCI, FANCL, FANCM and any alternatively spliced analogues. FANCA can also be referred to as: Fanconi Anemia Complementation Group A, FANCH, FACA, FAA, Fanconi Anemia Type 1, FA-H, FA1, FAH and FA. FANCC can also be referred to as: Fanconi Anemia Complementation Group C, FACC, FAC, Fanconi Anemia Group C Protein, and FA3. FANCD2 is also referred to as Fanconi Anemia Complementation Group D2, Fanconi Anemia Group D2 Protein, FANCD, FA-D2, FAD2, FA4. FANCE can also be referred to as: Fanconi Anemia Complementation Group E, Fanconi Anemia Group E Protein, FACE and FAE. FANCF can also referred be to as: Fanconi Anemia Complementation Group F, Fanconi Anemia Group F Protein, FACF and FAF. FANCG can also be referred to as: Fanconi Anemia Complementation Group G, Fanconi Anemia Group G Protein, DNA Repair Protein XRCC9, XRCC9 Truncated Fanconi Anemia Group G Protein, FACG and FAG. FANCI can also be referred to as: Fanconi Anemia Complementation Group I, Fanconi Anemia Group I Protein, KIAA1794 and FACI. FANCL can also be referred to as: Fanconi Anemia Complementation Group L, Fanconi Anemia Group L Protein, RING-Type E3 Ubiquitin Transferase FANCL, PHD Finger Protein 9, FAAP43, PHF9, E3 Ubiquitin-Protein Ligase FANCL and POG. FANCM can also be referred to as: Fanconi Anemia Complementation Group M, Fanconi Anemia Group M Protein, ATP-Dependent RNA Helicase FANCM, KIAA1596, Protein Hef Ortholog, FAAP250 and Protein FACM. FANCA comprises Homo sapiens FANCA encoded by amino acid sequences and nucleotide sequences according to NCBI accession numbers: NP_000126 and NM_000135.3. FANCC comprises Homo sapiens FANCC encoded by amino acid sequences and nucleotide sequences according to NCBI accession numbers: NP_000127 and NM_000136.2. FANCD2 comprises Homo sapiens FANCD2 encoded by amino acid sequences and nucleotide sequences according to NCBI accession numbers: NP_149075, NM_033084.4, NP_001306913 and NM_001319984.1. FANCE comprises Homo sapiens FANCE encoded by amino acid sequences and nucleotide sequences according to NCBI accession numbers: NP_068741 and NM_021922.2. FANCF comprises Homo sapiens FANCF encoded by amino acid sequences and nucleotide sequences according to NCBI accession numbers: NP_073562 and NM_022725.3. FANCG comprises Homo sapiens FANCG encoded by amino acid sequences and nucleotide sequences according to NCBI accession numbers: NP_004620 and NM_004629.1. FANCI comprises Homo sapiens FANCI encoded by amino acid sequences and nucleotide sequences according to NCBI accession numbers: NP_001106849 and NM_001113378.1. FANCL comprises Homo sapiens FANCL encoded by amino acid sequences and nucleotide sequences according to NCBI accession numbers: NP_001108108, NM_001114636.1, NP_060532 and NM_018062.3. FANCM comprises Homo sapiens FANCM encoded by amino acid sequences and nucleotide sequences according to NCBI accession numbers: NP_065988 and NM_020937.3.

The term “replication stress gene” refers to any gene that is induced or activated upon exposure of a cell increased DNA replication, increased initiation of replication (i.e., entry into S phase of cell cycle) increased mitosis, increased cell proliferation, increased DNA damage, excessive compacting of chromatin, over-expression of oncogenes or combinations thereof, and mediate the response to the stress, such as a stalled replication fork. Replication stress genes include, but are not limited to, the following genes: ATR, CHEK1 and any alternatively spliced analogues.

The term “oncogenic driver gene” or “oncogene” refers to any gene that when activated, over-expressed or otherwise increased in activity or abundance, leads to increased one or more hallmarks of tumor growth or cancer in a cell. Oncogenic driver genes include, but are not limited to, the following genes: CCNE1, KRAS, MYC, MYCN, MYCL, PI3KCA, CDK12 and any alternatively spliced analogues. In addition, negative regulators of oncogenic drivers, such as FBXW7, can also be viewed as oncogenic if mutation results in loss-of-function or reduced function. CCNE1 can also be referred to as: Cyclin E1, CCNE, G1/S-Specific Cyclin E1, Cyclin Es, Cyclin Et and PCCNE1. KRAS can also be referred to as KRAS Proto-Oncogene GTPase, V-Ki-Ras2 Kirsten Rat Sarcoma Viral Oncogene Homolog, V-Ki-Ras2 Kirsten Rat Sarcoma Viral Oncogene Homolog, Kirsten Rat Sarcoma Viral Proto-Oncogene, Cellular C-Ki-Ras2 Proto-Oncogene, Transforming Protein P21, C-Kirsten-Ras Protein, KRAS2A, K-RAS2B, K-RAS4A, K-RAs4B, K-Ras, KRAS1, C-Ki-Ras, K-Ras 2, C-K-RAS, CFC2, RALD, NS3 and NS. Myc can also be referred to as: C-Myc, MYC Proto-Oncogene BHLH Transcription Factor, V-Myc Avian Myelocytomatosis Viral Oncogene Homolog, Class E Basic Helix-Loop-Helix Protein 39, Proto-Oncogene C-Myc, BHLHe39, Avian Myelocytomatosis Viral Oncogene Homolog, Myc Proto-Oncogene Protein, MRTL and MYCC. MYCN can also be referred to as: N-MYC, MYCN Proto-Oncogene BHLH Transcription Factor, V-Myc Avian Myelocytomatosis Viral Oncogene Neuroblastoma Derived Homolog, Class E Basic Helix-Loop-Helix Protein 37, BHLHe37, NMYC, Neuroblastoma-Derived V-Myc Avian Myelocytomatosis Viral Related Oncogene, N-Myc Proto-Oncogene Protein, Neuroblastoma Myc Oncogene, Oncogene NMYC and ODED. MYCL can also be referred to as MYCL Proto-Oncogene, BHLH Transcription Factor, Class E Basic Helix-Loop-Helix Protein 38, Myc-Related Gene From Lung Cancer, Protein L-Myc-1, and v-Myc Avian Myelocytomatosis Viral Oncogene Homolog 1 Lung. PIK3CA can also be referred to as: Phosphatidylinositol-4,5-Bisphosphate 3-Kinase Catalytic Subunit Alpha, PI3-Kinase Subunit Alpha, Phosphoinositide-3-Kinase Catalytic Alpha Polypeptide, Serine/Threonine Protein Kinase PIK3CA, PI3K-Alpha, PI3-Kinase P110 Subunit Alpha, P110-Alpha, PI3Kalpha, P110alpha, CLOVE, MCMTC, MCAP, CWS5, PI3K and MCM. CDK12 can also be referred to as: Cyclin Dependent Kinase 12, Cyclin-Dependent Kinase 12, Cdc2-Related Kinase Arginine/Serine-Rich, Cell Division Cycle 2-Related Protein Kinase 7, Cell Division Protein Kinase 12, CDC2-Related Protein Kinase 7, CRKRS, CRK7, CDC2 Related Protein Kinase 7, Cyclin-Dependent Kinase 12, KIAA0904, HCDK12 and CRKR. CCNE1 comprises Homo sapiens CCNE1 encoded by amino acid sequences and nucleotide sequences according to NCBI accession numbers: NP_001229 and NM_001238.3. KRAS comprises Homo sapiens KRAS encoded by amino acid sequences and nucleotide sequences according to NCBI accession numbers: NP_203524 and NM_033360.3. MYC comprises Homo sapiens MYC encoded by amino acid sequences and nucleotide sequences according to NCBI accession numbers: NP_002458, NM_002467.5 and ABW69847. MYCN comprises Homo sapiens MYCN encoded by amino acid sequences and nucleotide sequences according to NCBI accession numbers: NP_005369 and NM_005378.5. MYCL comprises Homo sapiens MYCL encoded by amino acid sequences and nucleotide sequences according to NCBI accession numbers: NM_001033082 and NP_001028254.2. PIK3CA comprises Homo sapiens PIK3CA encoded by amino acid sequences and nucleotide sequences according to NCBI accession numbers: NM_006218 and NP_006209.2. CDK12 comprises Homo sapiens CDK12 encoded by amino acid sequences and nucleotide sequences according to NCBI accession numbers: NM_016507 and NP_057591.2.

The term “homologous recombination gene” refers to a gene that either directly or indirectly promotes, activates or is important for homologous recombination in cells. Homologous recombination genes include, but are not limited to, genes involved in double strand break repair (e.g., BRCA1, BRCA2, FANCN and RAD51C).

The term “replication stress” refers to stalled replication forks, genomic instability, increased mutation and/or mutation rate, activation of DNA damage repair pathways, activation of the DNA damage response (DDR), activation or increased expression of replication stress gene(s), or combinations thereof.

The term “high levels of replication stress” refers to tumors that exhibit increased levels of stalled replication forks, genomic instability, mutation and/or mutation rate, activation of DNA damage repair pathways, activation of the DNA damage response (DDR), activation or expression of replication stress gene(s), or combinations thereof compared to at least one reference sample (e.g., tumor cells from other individuals or normal non-tumor cells).

The term “external inducer of replication stress” refers to any agent that causes increased stalled replication forks, increased genomic instability, increased mutation and/or mutation rate, activation of DNA damage repair pathways, activation of the DNA damage response (DDR), activation or increased expression of replication stress gene(s), or combinations thereof. Examples of inducers of replication stress include, but are not limited to, genotoxic chemotherapeutic agents (e.g., gemcitabine and other nucleoside analogs, alkylating agents such as temozolomide, cisplatin, mitomycin C and others, topoisomerase inhibitors such as camptothecin and etoposide and others), inhibitors of ATR and inhibitors of PARP). External inducers of cell stress include agents that reduce the concentration of nucleotides in a cell (e.g., ribonucleotide reductase inhibitors such as hydroxyurea, also known as, hydroxycarbamide, and the like).

The term “chemotherapy” refers to administration of any genotoxic agent (e.g., DNA damaging agent), including conventional or non-conventional chemotherapeutic agents, for the treatment or prevention of cancer. Chemotherapeutic agents include agents that have been modified, (e.g., fused to antibodies or other targeting agents). Examples of chemotherapeutic agents include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine, mitomycin C), antitumor antibiotics (e.g., daunorubicin, doxorubicin, idarubicin, epirubicin, mitoxantrone, bleomycin, plicamycin, dactinomycin), taxanes (e.g., paclitaxel, nab-paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, nelarabine, gemcitabine, 5-flurouracil), topoisomerase inhibitors (e.g., topotecan, irinotecan, SN-38, CPT-11), hypomethylating agents (e.g., azacitidine and decitabine), proteasome inhibitors (e.g., bortezomib), epipodophyllotoxins (e.g., etoposide and teniposide), DNA synthesis inhibitors (e.g., hydroxyurea), and vinca alkaloids (e.g., vincristine, vindesine, vinorelbine, and vinblastine). Chemotherapeutic agents includes DNA intercalating agents (e.g., pyrrolobenzodiazepines).

Abbreviations used in this application include the following: Chk1 (Checkpoint Kinase 1), DDR (DNA Damage Response), TS (Tumor Suppressor), NGS (next generation sequencing), and CCNE1 (Cyclin E1), HGSOC (high grade serous ovarian cancer), HPV (human papillomavirus), and CRC (colorectal cancer).

Recitation of ranges herein includes the recited endpoints and all points there between.

It must be noted that, as used in the specification and the appended claims, the singular forms “a,” “an” and “the” include plural referents unless the context clearly dictates otherwise.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Inhibition of Chk1 Causes Death of Cancer Cells In Vitro

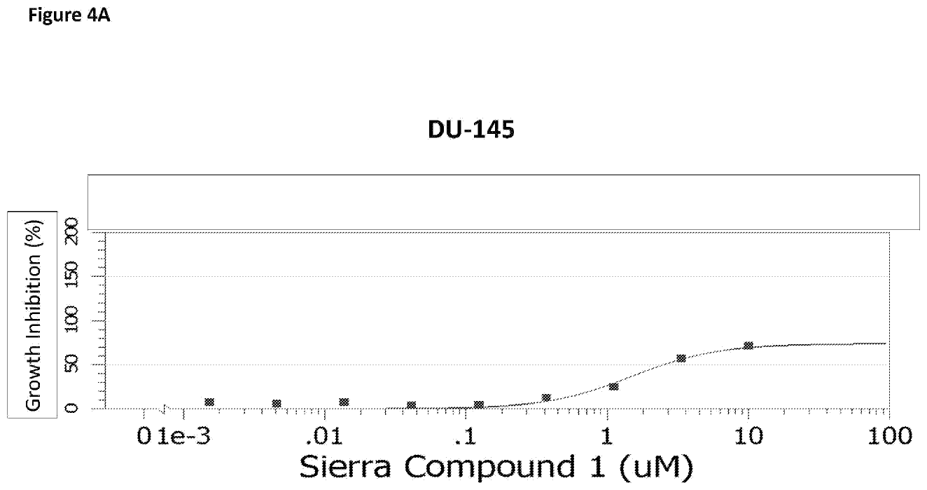

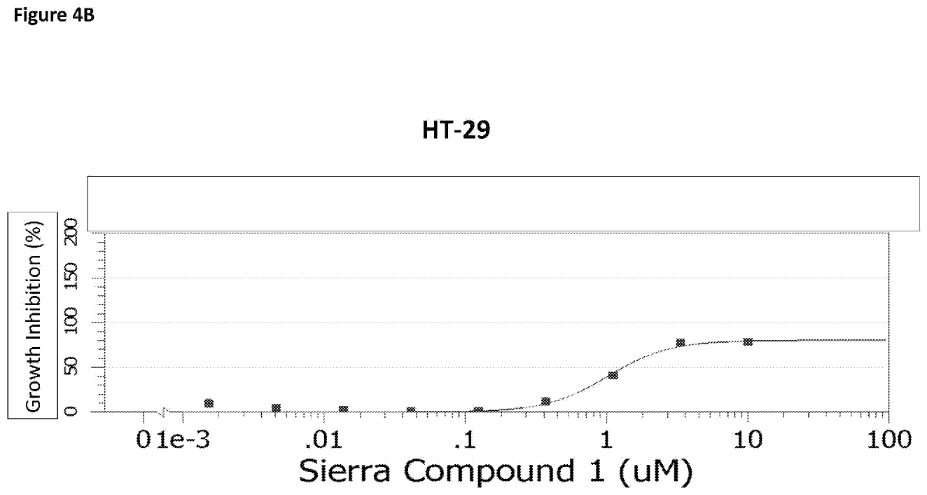

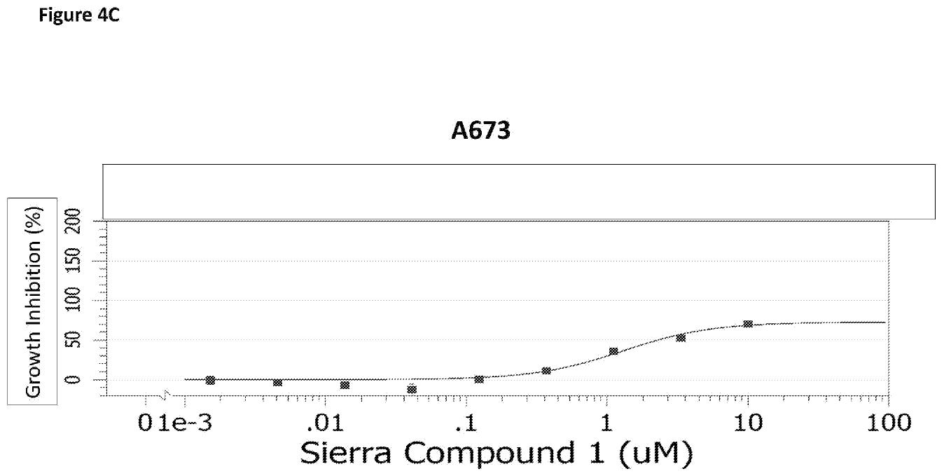

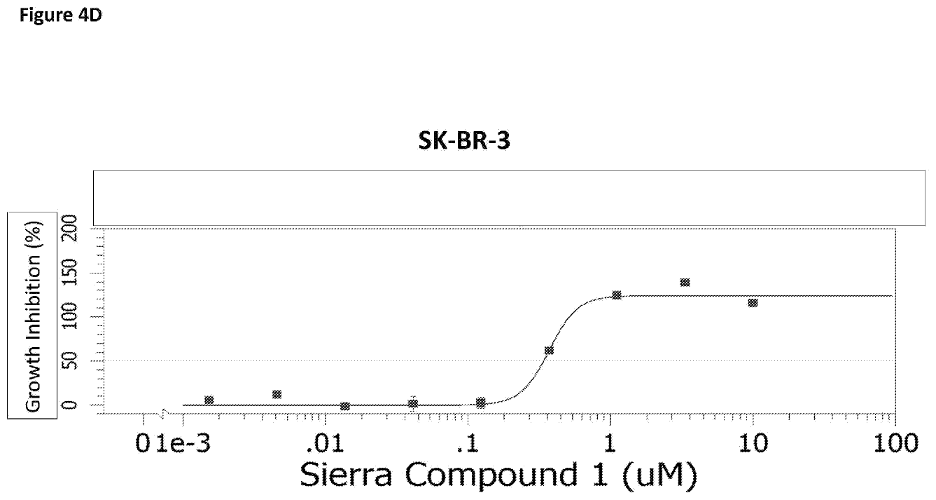

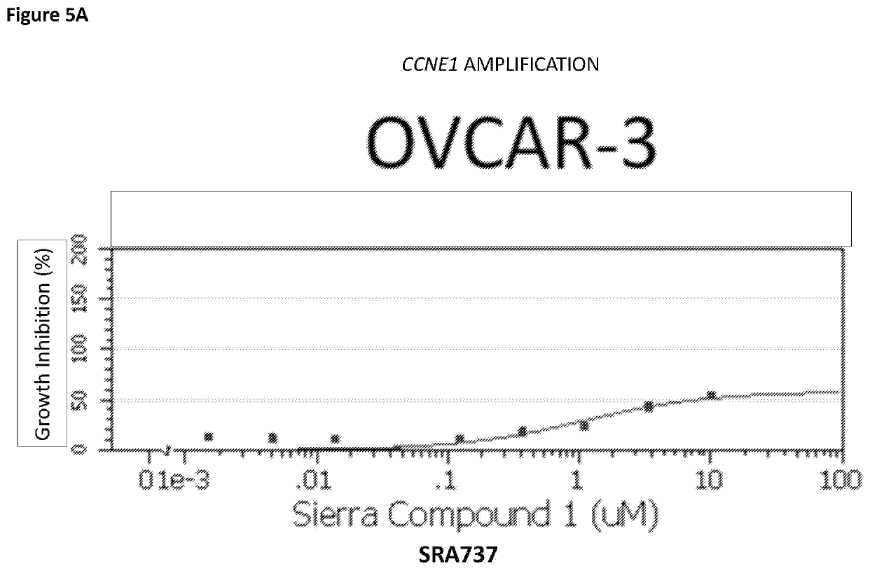

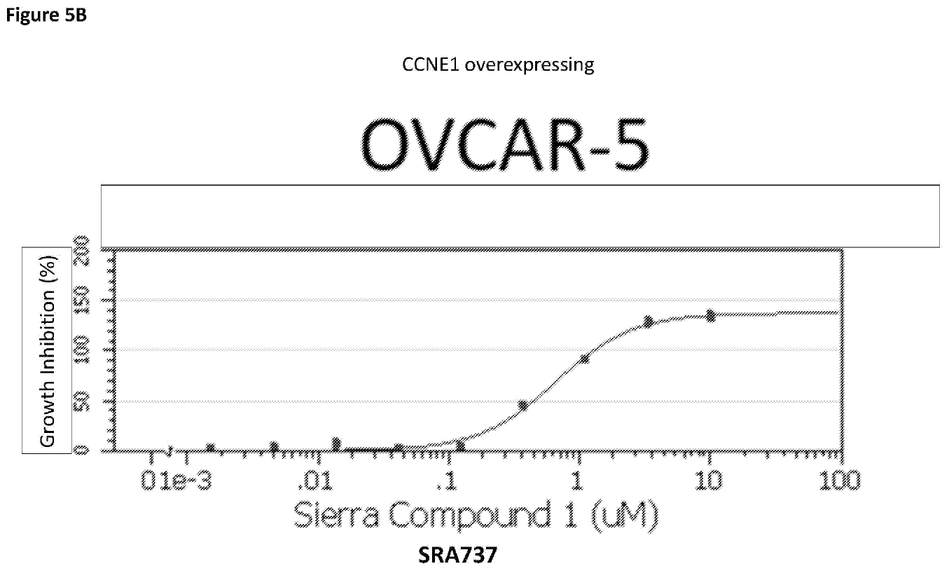

In cancer cells, replication stress induced by oncogenes (e.g., MYC and RAS oncogenes) and/or loss of function in tumor suppressors (e.g., TP53) results in persistent DNA damage and genomic instability, a trigger of the DDR network. To confirm that targeted inhibition of the component of the DDR network, Chk1, is synthetically lethal to cancer cells and thus may have utility as a therapy in a range of tumor indications particularly in tumors with high rates of replication stress and genomic instability, various cancer cell lines with known genetic alterations expected to confer sensitivity to Chk1 inhibition were incubated with the Chk1 inhibitor, SRA737 in vitro (FIGS. 4 and 5). The SK-BR-3, OVCAR-3, OVCAR-5, HT-29, DU-145, and A673 demonstrated sensitivity to Chk1 inhibition. SK-BR-3 is a breast cancer cell line harboring a deleterious mutation is TP53 and amplification in c-MYC. HT-29 is a colorectal cancer (CRC) cell line harboring a deleterious mutation in the tumor suppressor gene, adenomatous polyposis coli (APC), an amplification in c-MYC, and an activation mutation in the oncogenes, BRAF and PIK3CA. DU-145 is metastatic prostate cancer cell line harboring a deleterious mutation in the tumor suppressors TP53 and RB1, an amplification in KRAS, and alterations of the DNA damage repair genes TP53B1, MLH1 and MSH3. A673 is a rhabdomyosarcoma cell line harboring genetic alterations in the tumor suppressor genes, CDKN2A and CDKN2B and genetic alterations in the DNA damage repair genes BRCA1, Rad50 and Rad51D. OVCAR-3 is an ovarian cancer cell line harboring a deleterious mutations in the tumor suppressors TP53 and RB1 and an amplification of CCNE1. OVCAR-5 is an ovarian cancer cell line harboring an activation mutation in KRAS, exhibits over expression of CCNE1 and harbors genetic alterations in the tumor suppressor genes, CDKN2A and CDKN2B. The results of these in vitro experiments confirm that cancer cells with genetic alterations in tumor suppressor genes, oncogenes, and/or DNA damage repair genes are sensitive to inhibition of Chk1.

### Example 2: Inhibition of Chk1 Reduces Tumor Growth of HGSOC Cells in a Xenograft Model

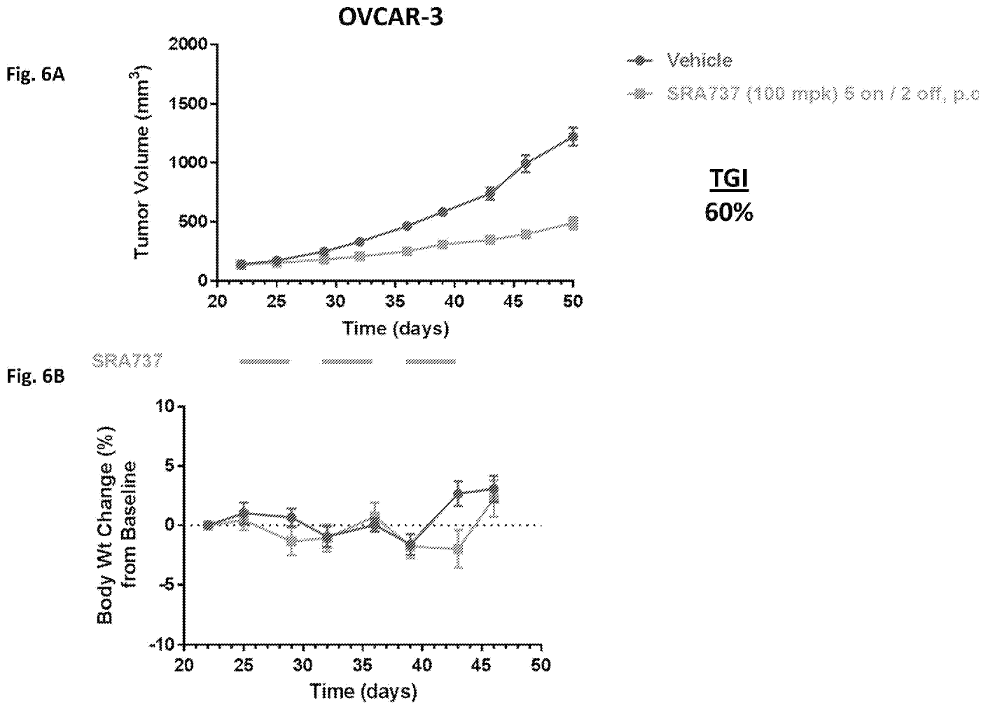

To confirm the anti-tumor activity of Chk1 inhibition in tumors with gene overexpression of CCNE1 in vivo, an OVCAR-3 xenograft model of high grade serous ovarian cancer (HGSOC) was tested with SRA737. The OVCAR-3 tumor cell line was derived from a platinum insensitive patient and harbors an amplification of CCNE1 (encoding cyclin E) and mutation in TP53 (encoding p53), two genomic alterations described herein to contribute to Chk1 inhibitor sensitivity. OVCAR-3 tumor-bearing mice were treated with SRA737 at a dose of 100 mg/kg for 3 cycles in a 5 days on, and 2 days off schedule. On Day 46 of treatment, a significant tumor growth inhibition of 60% relative to vehicle treated mice was observed (P<0.0001), as shown in FIG. 6A. Treatment with SRA737 was well tolerated as demonstrated by minimal body weight loss and resulted in tumor growth inhibition (FIG. 6B). These results confirm that cancer cells harboring overexpression of CCNE1 gene are sensitive to Chk1 inhibition.

### Example 3: Inhibition of Chk1 Reduces Tumor Growth of Cells Proficient in Homologous Recombination

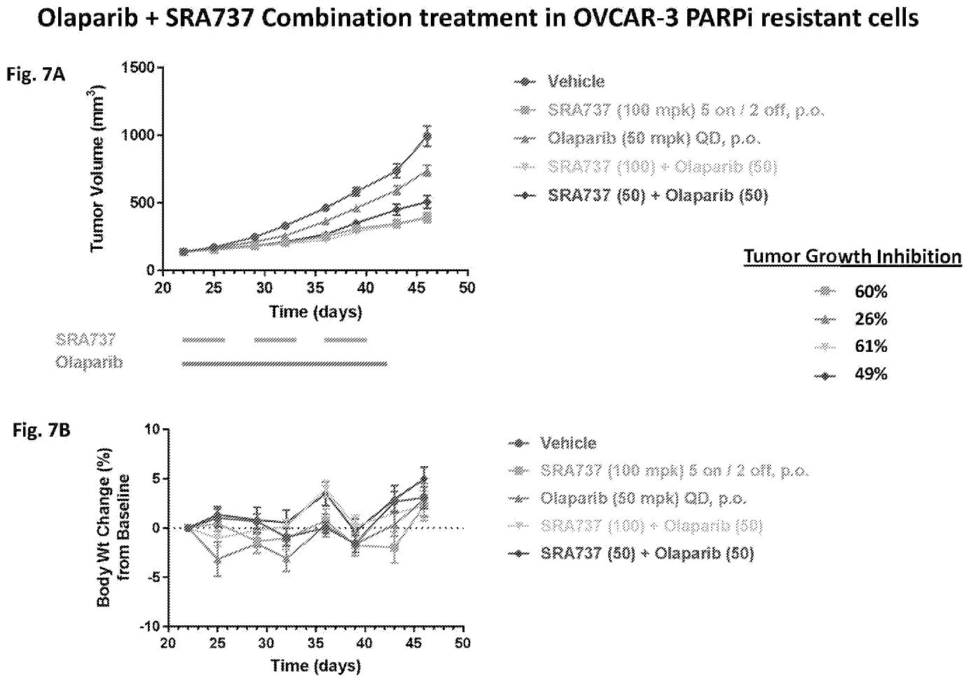

To confirm the anti-tumor activity of Chk1 inhibition in cells that are proficient in homologous recombination and resistant to inhibition of PARP, SRA737 and the PARP inhibitor, Olaparib, were tested in the OVCAR-3 xenograft model (FIG. 7). Treatment with SRA737 at a dose of 100 mg/kg significantly reduced tumor growth as compared to Olaparib, confirming that PARP resistant cells proficient in homologous recombination are sensitive to Chk1 inhibition (FIG. 7A), whereas body weight was not significantly altered, demonstrating that SRA737 is well-tolerated at the administered doses (FIG. 7B).

### Example 4: Phase ½ Clinical Study to Confirm Efficacy of Chk1 Inhibition in Select Tumors with Genetic Alterations that Confer Chk1 Sensitivity

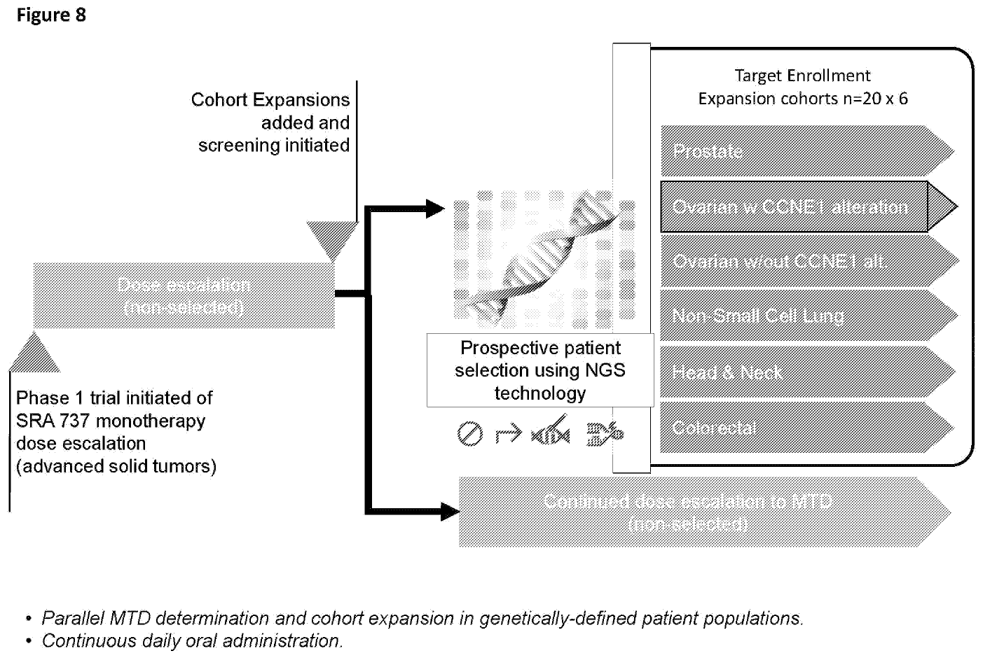

An in-human dose escalation study enriched in dose expansion cohorts with prospectively-selected genetically-defined subjects in tumor types that are known to have a high prevalence of genomic alterations expected to sensitize the tumor to Chk1 inhibition is conducted to confirm the methods of treatment and patient selection strategies disclosed herein. Dose Escalation and Expansion Cohorts phases can occur in parallel (FIG. 8). The Cohort Expansion Phase consists of 6 indication-specific expansion cohorts of approximately 20 prospectively-selected genetically-defined subjects each. The cohorts are subjects with previously treated metastatic colorectal cancer [CRC], high grade serous ovarian cancer [HGSOC] without CCNE1 gene amplification, HGSOC with CCNE1 gene amplification (or alternative genetic alteration with similar functional effect), metastatic castration-resistant prostate cancer [mCRPC], advanced non-small cell lung cancer [NSCLC], and squamous cell carcinoma of the head and neck [HNSCC], or squamous cell carcinoma of the anus [SCCA].

Subjects have tumor tissue or ctDNA evidence that their tumor harbors a combination of mutations which are expected to confer sensitivity to Chk1 inhibition. Subjects are selected based on prospective, tumor tissue genetic profiling using NGS.

Expansion cohort subjects have tumors that harbor genomic alterations expected to confer sensitivity to Chk1 inhibition in a minimum of two of the following categories (a)-(e):
a. Key tumor suppressor genes regulating G1 cell cycle progression/arrest such as RB1, TP53, etc. For patients with NHSCC or SCCA, positive HPV status is also considered for eligibility.
b. The DDR pathway including ATM, BRCA1, and BRCA2. For patients with CRC, mismatch repair (MMR) genetic alterations and/or high microsatellite instability are also considered for eligibility.
c. Genetic indicators of replicative stress such as gain of function/amplification of Chk1 or ATR or other related gene.
d. Oncogenic drivers such as MYC, KRAS, etc.
e. CCNE1 gene amplification (or alternative genetic alteration with similar functional effect) is required for the CCNE1 gene amplification-specific HGSOC cohort.

Subjects must meet one of the following criteria (a-e):
(a) Metastatic CRC
   i. Histologically and/or cytologically confirmed CRC
   ii. Must have received at least 1 prior regimen for advanced/metastatic disease
(b) HGSOC
   i. Histologically confirmed high grade serous ovarian, fallopian tube or primary peritoneal cancer
   ii. Recurrent platinum-intolerant subjects, or those with platinum-resistant disease, defined as radiological evidence of disease progression within 6 months of the last receipt of platinum-based chemotherapy. Patients with platinum refractory disease (as defined by the European Society for Medical oncology [ESMO] Guidelines) are not eligible.
(c) Advanced NSCLC
   i. Locally advanced and recurrent or metastatic, histologically confirmed NSCLC
   ii. Must have received at least 1 prior regimen for advanced/metastatic disease
(d) mCPRC
   i. Histologically or cytologically confirmed adenocarcinoma of the prostate that has progressed after androgen deprivation therapy
(e) HNSCC or SCCA
   i. Histologically confirmed HNSCC from any primary site, or SCCA
   ii. For HNSCC: locally advanced disease (i.e., persistent or progressive disease following curative-intent radiation, and not a candidate for surgical salvage due to incurability or morbidity), or metastatic disease
   iii. For SCCA: locally advanced disease or metastatic disease for which no curative intent therapy is available.
   iv. Subjects have received at least 1 prior regimen for advanced/metastatic disease.

Subjects must have measurable disease (per Response Evaluation Criteria in Solid Tumors, version 1.1 [RECIST v1.1]) or, for mCRPC, evaluable disease per any of the following:

Measurable disease per RECIST v1.1; increasing prostate specific antigen (PS); or circulating tumor cell (CTC) count of 5 or more cells per 7.5 ml of blood.

Enrollment to the Dose Escalation and Expansion Cohorts may occur in parallel. A subject that qualifies for the Cohort Expansion Phase will be enrolled into an Escalation Cohort whenever possible. Any such subject will be considered to have enrolled in both phases simultaneously. For dose escalation, SRA737 is administered orally on a continuous daily dosing of each 28-day cycle. Cohorts consisting initially of a single subject will receive escalating doses of SRA737, starting in Cohort 1 with 20 mg/day administered orally on a continuous daily dosing schedule in 28-day cycles. The dose is escalated until the MTD has been identified, unless determined otherwise by the Sponsor in consultation with the Chief Investigator, for example, if an alternative schedule is pursued instead. Once a SRA737-related, Grade 2 toxicity is observed in a dose escalation cohort during Cycle 1, that cohort is expanded to 3 to 6 subjects, and subsequent dose level cohorts will follow a rolling 6 design.

The results of this study confirm the efficacy of Chk1 inhibitors for the treatment of tumors with known genetic alterations expected to confer sensitivity to Chk1 inhibition

### Example 5: Phase 1/2 Clinical Study to Confirm Efficacy of Chk1 Inhibition in Select Tumors with Genetic Alterations that Confer Chk1 Sensitivity Comprising Deleterious Mutations in Tumor Suppressor Genes

A clinical trial is conducted as described above in Example 4 to confirm the efficacy of SRA737 in prospectively-selected genetically-defined subjects having a tumor harboring genomic aberrations expected to sensitize the tumor to Chk1 inhibition, wherein the genomic aberrations comprise at least one tumor suppressor gene regulating G1 cell cycle progression/arrest such as RB1, TP53, etc. (for patients with NHSCC or SCCA, positive HPV status is also considered for eligibility), and at least one of the genetic alterations for one of the following categories (a)-(d):
(a) The DDR pathway including ATM, BRCA1, and BRCA2. For patients with CRC, mismatch repair (MMR) genetic alterations and/or high microsatellite instability are also considered for eligibility.
(b) Genetic indicators of replicative stress such as gain of function/amplification of CHEK1 or ATR or other related gene.
(c) Oncogenic drivers such as MYC, KRAS, etc.
(d) CCNE1 gene amplification (or alternative genetic alteration with similar functional effect) is required for the CCNE1 gene amplification-specific HGSOC cohort.

The results of this study confirm the efficacy of Chk1 inhibitors for the treatment of tumors with deleterious mutations in tumor suppressor genes and at least one other genetic alteration expected to confer sensitivity to Chk1 inhibition

### Example 6: Phase 1/2 Clinical Study to Confirm Efficacy of Chk1 Inhibition in Select Tumors with Genetic Alterations that Confer Chk1 Sensitivity Comprising Deleterious Mutations in Tumor Suppressor Genes

A clinical trial is conducted as described above in Example 4 to confirm the efficacy of SRA737 in prospectively-selected genetically-defined subjects. Subjects have a tumor that harbors a genomic alteration expected to confer sensitivity to Chk1 inhibition in a minimum of one of the following categories (a)-(e):
(a) Key tumor suppressor genes regulating G1 cell cycle progression/arrest such as RB1, TP53, etc. For patients with NHSCC or SCCA, positive HPV status is also considered for eligibility.
(b) The DDR pathway including ATM, BRCA1, and BRCA2. For patients with CRC, mismatch repair (MMR) genetic alterations and/or high microsatellite instability are also considered for eligibility.
(c) Genetic indicators of replicative stress such as gain of function/amplification of Chk1 or ATR or other related gene.
(d) Oncogenic drivers such as MYC, KRAS, etc.
(e) CCNE1 gene amplification (or alternative genetic alteration with similar functional effect) is required for the CCNE1 gene amplification-specific HGSOC cohort.

Subjects are co-administered SRA737 PO daily at a dose of 40 mg/kg-800 mg/kg for at least one 28 day cycle and gemcitabine at a dose of 50-500 mg/m² IV for at least one day of a 28 day cycle.

The results of this study confirm the efficacy of Chk1 inhibitors in combination with a genotoxic agent for the treatment of tumors at least one known genetic alterations expected to confer sensitivity to Chk1 inhibition

What is claimed is:

1. A method of treating a tumor in an individual having a cancer, the method comprising:
administering a Chk1 inhibitor to the individual, wherein the tumor is identified as having genetic alterations that confer high levels of replication stress and thereby sensitivity to the Chk1 inhibitor by synthetic lethality; and wherein the genetic alterations are at least two of property a, property b, property c, or property d wherein:
property a is a gain of function mutation, amplification or overexpression of at least one oncogenic driver gene or other gene implicated in Chk1 pathway sensitivity;
property b is a loss of function or deleterious mutation in at least one DNA damage repair (DDR) pathway gene implicated in Chk1 pathway sensitivity;
property c is a gain of function mutation or amplification of at least one replication stress gene; and
property d is a deleterious mutation in a tumor suppressor (TS) gene implicated in Chk1 pathway sensitivity.

2. The method of method of claim 1, wherein the genetic alterations are the property d and at least one of the property a, property b or property c.

3. The method of claim 1 or claim 2, further comprising determining whether or not the tumor comprises the property a, property b, property c or property d.

4. The method of claim 3, wherein the property a, property b, property c, or property d are determined by using Next-Generation Sequencing (NGS), by immunohistochemistry, by mass spectrometry (MS), by liquid chromatograph mass spectrometry (LC-MS), by quantitative PCR, by RNA sequencing (RNAseq) or by fluorescence activated cell sorting (FACS) analysis.

5. The method of claim 4, wherein the property a, property b, property c, or property d are determined using NGS.

6. The method of any one of the above claims, wherein the tumor suppressor gene is RB1, TP53 or ATM.

7. The method of any one of the above claims, wherein the tumor suppressor gene is RAD50, FBXW7, PARK2, CDKN2A or CDKN2B.

8. The method of any one of claims 1-5 wherein property d is established by establishing positivity for human papillomavirus (HPV).

9. The method of claim 8, wherein the cancer is a squamous cell carcinoma.

10. The method of claim 9, wherein the squamous cell carcinoma is head and neck squamous cell carcinoma, cervical cancer or anogenital squamous cell carcinoma.

11. The method of any one of the above claims, wherein the oncogenic driver gene is MYC, MYCN, or CCNE1.

12. The method of any one of the above claims, wherein the DDR pathway gene is ATM, BRCA1, BRCA2 or an FA pathway gene.

13. The method of any one of the above claims, wherein the DDR pathway gene is MRE11A or ATR.

14. The method of any one of the above claims, wherein property b is established by establishing a microsatellite instability or a deficiency in mismatch repair (MMR).

15. The method of claim 14, wherein the cancer is colorectal cancer (CRC) or endometrial cancer.

16. The method of any one of the above claims, wherein the replication stress gene is ATR or CHEK1.

17. The method of any one of the above claims, further comprising chemotherapy, a treatment comprising administering an antibody, antibody fragment, antibody drug conjugate or radiation treatment.

18. The method of any one of the above claims, further comprising administering an external inducer of replication stress.

19. The method of any one of the above claims, further comprising administering gemcitabine, cisplatin, hydroxyurea, a ribonucleotide reductase inhibitor, etoposide, SN-38/CPT-11, mitomycin C, an inhibitor of ATR, an inhibitor of PARP or combinations thereof.

20. The method of claim 19, further comprising administering gemcitabine.

21. The method of any one of the above claims, wherein the individual has a cancer selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

22. The method of any one of the above claims, wherein the Chk1 inhibitor is SRA737.

23. A method of treating a tumor in an individual having a cancer, the method comprising:
administering a Chk1 inhibitor and a genotoxic agent that confers increased levels of replication stress to the individual, wherein the tumor is identified as having at least one genetic alteration that confers high levels of replication stress and thereby sensitivity to the Chk1 inhibitor by synthetic lethality; and wherein the genetic alteration is at least one of property a, property b, property c, or property d wherein:
property a is a gain of function mutation, amplification or overexpression of at least one oncogenic driver gene or other gene implicated in Chk1 pathway sensitivity;
property b is a loss of function or deleterious mutation in at least one DNA damage repair (DDR) pathway gene implicated in Chk1 pathway sensitivity;
property c is a gain of function mutation or amplification of at least one replication stress gene implicated in Chk1 pathway sensitivity; and
property d is a deleterious mutation in a tumor suppressor (TS) gene implicated in Chk1 pathway sensitivity.

24. The method of claim 23, further comprising determining whether or not the tumor comprises at least one of the property a, property b, property c, or property d.

25. The method of claim 24, wherein the property a, property b, property c, or property d are determined by using Next-Generation Sequencing (NGS), by immunohistochemistry, by mass spectrometry (MS), by liquid chromatograph mass spectrometry (LC-MS), by quantitative PCR, by RNA sequencing (RNAseq) or by fluorescence activated cell sorting (FACS) analysis.

26. The method of claim 25, wherein at least one of the property a, property b, property c, or property d is determined using Next-Generation Sequencing (NGS).

27. The method of any one of claims 23-26, wherein the tumor suppressor gene is RB1, TP53 or ATM.

28. The method of any one of claims 23-27, wherein the tumor suppressor gene is RAD50, FBXW7 or PARK2.

29. The method of any one of claims 23-26, wherein property d is established by establishing positivity for HPV.

30. The method of claim 29, wherein the cancer is a squamous cell carcinoma.

31. The method of claim 30, wherein the squamous cell carcinoma is head and neck squamous cell carcinoma, cervical cancer, anogenital squamous cell carcinoma.

32. The method of any one of claims 23-31, wherein the oncogenic driver gene is MYC, MYCL, MYCN or CCNE1.

33. The method of any one of claims 23-32, wherein the DDR pathway gene is ATM, BRCA1, BRCA2 or an FA pathway gene.

34. The method of any one of claims 23-33, wherein the DDR pathway gene is MRE11A or ATR.

35. The method of any one of claims 23-34, wherein property b is established by establishing microsatellite instability or a deficiency in mismatch repair (MMR).

36. The method of claim 35, wherein the cancer is colorectal cancer (CRC) or endometrial cancer.

37. The method of any one of claims 23-36, wherein the replication stress gene is ATR or CHK1.

38. The method of any one of claims 23-37, further comprising chemotherapy, a treatment comprising administering an antibody, antibody fragment, antibody drug conjugate or radiation treatment.

39. The method of any one of claims 23-38, further comprising administering an external inducer of replication stress.

40. The method of any one of claims 23-39, wherein the genotoxic agent is gemcitabine, hydroxyurea, cisplatin, a ribonucleotide reductase inhibitor, etoposide, SN-38/CPT-11, mitomycin C, an inhibitor of ATR, an inhibitor of PARP or combinations thereof.

41. The method of claim 40, wherein the genotoxic agent is gemcitabine.

42. The method of any one of the above claims, wherein the individual has a cancer selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

43. The method of any one of claims 23-42, wherein the Chk1 inhibitor is SRA737.

44. A method of treating a tumor in an individual having cancer, the method comprising administering a Chk1 inhibitor to the individual, wherein the tumor, germline or combinations thereof is characterized by having wild type BRCA1 or BRCA2 and is resistant or refractory to platinum based chemotherapy.

45. A method of treating a tumor in an individual having cancer, the method comprising administering a Chk1 inhibitor to the individual, wherein the tumor has been previously treated with a PARP inhibitor on the basis of at least one mutation in a homologous recombination gene.

46. The method of claim 44 or claim 45, wherein the tumor is further characterized by a mutation, optionally a deleterious mutation, in a tumor suppressor gene.

47. The method claim 46, wherein the tumor suppressor gene is RB1, TP53, ATM, RAD50, FBXW7 or PARK2.

48. The method of claim 46 or 47, further comprising determining whether or not the tumor comprises the tumor suppressor gene mutation.

49. The method of any one of claims 44-48, wherein the tumor is further characterized by CCNE1 gene overexpression; and wherein the CCNE1 gene overexpression is at least one of overexpression of Cyclin E1 (CCNE1), CCNE1 gene amplification, CCNE1 gene copy number gain, CCNE1 mRNA overexpression, Cyclin E protein overexpression, or combinations thereof.

50. The method of claim 46, wherein CCNE1 gene overexpression is increased CCNE1 mRNA levels, Cyclin E protein levels or combinations thereof compared to a at least one reference sample.

51. The method of any one of claims 44-50, wherein the CCNE1 gene overexpression is detected by immunohistochemistry (IHC), by mass spectrometry (MS) or by liquid chromatography mass spectrometry (LC-MS).

52. The method of any one of claims 44-51, wherein the CCNE1 gene overexpression is caused by CCNE1 gene amplification or alternative genetic alteration with similar functional effect.

53. The method of claim 52, wherein the CCNE1 gene amplification or alternative genetic alteration is detected by NGS.

54. The method of any one of claims 49-53, further comprising characterizing the CCNE1 gene overexpression, optionally by using NGS, by IHC, by mass spectrometry (MS), by liquid chromatograph mass spectrometry (LC-MS), by quantitative PCR, by RNAseq, by FACS analysis or by determination of CyclinE-CDK2 activity.

55. The method of claim 54, wherein the characterization of the CCNE1 gene overexpression is performed by detecting circulating RNA or circulating DNA.

56. The method of claim 54, wherein the determination of CyclinE-CDK2 activity is detecting phosphorylation of CyclinE-CDK2 substrates.

57. The method of claim 43-56, wherein the CyclinE-CDK2 substrate is MCM2, retinoblastoma protein (Rb), p27, p21, Smad3, CBP/p300, E2F-5, p220(NPAT) or FOXO1.

58. The method of any one of claims 44-57, further comprising chemotherapy, a treatment comprising administering an antibody, antibody fragment, antibody drug conjugate or radiation treatment.

59. The method of any one of claims 44-58, further comprising administering an external inducer of replication stress.

60. The method of any one of claims 44-59, further comprising administering gemcitabine, hydroxyurea, cisplatin, a ribonucleotide reductase inhibitor, etoposide, SN-38/CPT-11, mitomycin C, an inhibitor of ATR, an inhibitor of PARP or combinations thereof.

61. The method of claim of claim 60, further comprising administering gemcitabine.

62. The method of method of any one of claims 44-60, wherein the individual has a cancer selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma, anogenital squamous cell carcinoma, anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

63. The method of claim 62, wherein the cancer is ovarian cancer.

64. The method of claim 43-63, wherein the ovarian cancer is high grade serous ovarian cancer (HGSOC).

65. The method of claim 64, wherein the HGSOC is deficient in homologous recombination or proficient in homologous recombination.

66. The method of any one of claims 44-65, wherein the Chk1 inhibitor is SRA737.

67. A method of treating a tumor in an individual having colorectal cancer, wherein the tumor is characterized by microsatellite instability or having a mismatch repair deficiency, the method comprising administering a Chk1 inhibitor to the individual.

68. A method of treating a tumor in an individual having endometrial cancer, wherein the tumor is characterized by microsatellite instability or having a mismatch repair deficiency, the method comprising administering a Chk1 inhibitor to the individual

69. A method of treating a tumor in an individual having squamous cell carcinoma, wherein the individual is HPV positive, the method comprising administering a Chk1 inhibitor to the individual.

70. The method of claim 69, wherein the squamous cell carcinoma is head and neck squamous cell carcinoma, cervical cancer or anogenital squamous cell carcinoma.

71. The method of claim 69 or claim 70, wherein the tumor is further characterized by at least one of property a, property b, or property c, wherein:
property a is a gain of function mutation or amplification of at least one oncogenic driver gene or other gene implicated in Chk1 pathway sensitivity;
property b is a loss of function or deleterious mutation in at least one DNA damage repair (DDR) pathway gene implicated in Chk1 pathway sensitivity; and
property c is a gain of function mutation or amplification of at least one replication stress gene implicated in Chk1 pathway sensitivity.
